(19) United States
(12) Patent Application Publication
(10) Pub. No.: US 2025/0381007 A1
(43) Pub. Date: Dec. 18, 2025

(54) CAMERA-BASED DEEP LEARNING PREDICTION AND GUIDANCE FOR MEDICAL IMAGING PROTOCOLS

(71) Applicant: GE Precision Healthcare LLC, Waukesha, WI (US)

(72) Inventors: Sajith Rajamani, Bangalore (IN); Deepa Anand, Bengaluru (IN); Dattesh Dayanand Shanbhag, Bengaluru (IN); Krishna Seetharam Shriram, Bangalore (IN); Jessica Leigh Buzek, Waukesha, WI (US); Uday Damodar Patil, Bangalore (IN)

(21) Appl. No.: 18/743,638

(22) Filed: Jun. 14, 2024

(51) Int. Cl. A61B 90/00

(52) U.S. Cl. CPC A61B 90/361

(57) ABSTRACT

Systems or techniques that facilitate camera-based deep learning prediction and guidance for medical imaging protocols are provided. In various embodiments, a system can infer, via execution of a first deep learning neural network, a prescribed imaging protocol that is to be performed by a medical imaging scanner on a medical patient. In various aspects, the system can infer, via execution of a second deep learning neural network on a preparation image or video of the medical patient that is captured by a camera associated with the medical imaging scanner, whether or not the medical patient is prepared for the prescribed imaging protocol. In various instances, the system can, in response to an inference that the medical patient is not prepared for the prescribed imaging protocol, initiate an electronic guidance action that explains or shows how to make the medical patient prepared for the prescribed imaging protocol.

### TECHNICAL FIELD

The subject disclosure relates generally to medical imaging scanners, and more specifically to camera-based deep learning prediction and guidance for medical imaging protocols.

### BACKGROUND

A medical imaging scanner can perform a wide variety of imaging protocols on medical patients. Such protocol variety, when combined with the realities of clinical time limitations and widely varying technician experience levels, can lead to unacceptably high scanning error rates.

Accordingly, systems or techniques that can address one or more of these technical problems can be desirable.

### SUMMARY

The following presents a summary to provide a basic understanding of one or more embodiments. This summary is not intended to identify key or critical elements, or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatus or computer program products that facilitate camera-based deep learning prediction and guidance for medical imaging protocols are described.

According to one or more embodiments, a system is provided. The system can comprise a non-transitory computer-readable memory that can store computer-executable components. The system can further comprise a processor that can be operably coupled to the non-transitory computer-readable memory and that can execute the computer-executable components stored in the non-transitory computer-readable memory. In various embodiments, the computer-executable components can comprise a protocol component that can infer, via execution of a first deep learning neural network, a prescribed imaging protocol that is to be performed by a medical imaging scanner on a medical patient. In various aspects, the computer-executable components can comprise a preparation component that can infer, via execution of a second deep learning neural network on a preparation image or video of the medical patient that is captured by a camera associated with the medical imaging scanner, whether or not the medical patient is prepared for the prescribed imaging protocol. In various instances, the computer-executable components can comprise a guidance component that, in response to an inference that the medical patient is not prepared for the prescribed imaging protocol, can initiate an electronic guidance action that explains or shows how to make the medical patient prepared for the prescribed imaging protocol.

According to one or more embodiments, a computer-implemented method is provided. In various embodiments, the computer-implemented method can comprise inferring, by a device operatively coupled to a processor and via execution of a first deep learning neural network, a prescribed imaging protocol that is to be performed by a medical imaging scanner on a medical patient. In various aspects, the computer-implemented method can comprise inferring, by the device and via execution of a second deep learning neural network on a preparation image or video of the medical patient that is captured by a camera associated with the medical imaging scanner, whether or not the medical patient is prepared for the prescribed imaging protocol. In various instances, the computer-implemented method can comprise initiating, by the device and in response to an inference that the medical patient is not prepared for the prescribed imaging protocol, an electronic guidance action that explains or shows how to make the medical patient prepared for the prescribed imaging protocol.

According to one or more embodiments, a computer program product for facilitating camera-based deep learning prediction and guidance for medical imaging protocols is provided. In various embodiments, the computer program product can comprise a non-transitory computer-readable memory having program instructions embodied therewith. In various aspects, the program instructions can be executable by a processor to cause the processor to infer, via execution of a first deep learning neural network on a physician prescription corresponding to a medical patient or on a video feed depicting the medical patient, a prescribed imaging protocol that is to be performed by a magnetic resonance imaging (MRI) scanner on the medical patient. In various aspects, the program instructions can be further executable to cause the processor to infer, via execution of a second deep learning neural network on the video feed, whether or not an MRI coil position on the medical patient fails to match a requisite MRI coil position specified in the prescribed imaging protocol. In various instances, the program instructions can be further executable to cause the processor to cause, in response to an inference that the MRI coil position does not match the requisite MRI coil position, an actuatable light or laser associated with the MRI scanner to shine onto the body of the medical patient, thereby visibly lighting the requisite MRI coil position on the medical patient.

### DESCRIPTION OF THE DRAWINGS

FIG. 1 illustrates a block diagram of an example, non-limiting system that facilitates camera-based deep learning prediction and guidance for medical imaging protocols in accordance with one or more embodiments described herein.

FIG. 2 illustrates a block diagram of an example, non-limiting system including a prescription document and a preparation image or video that facilitates camera-based deep learning prediction and guidance for medical imaging protocols in accordance with one or more embodiments described herein.

FIG. 3 illustrates a block diagram of an example, non-limiting system including a first deep learning neural network and a prescribed imaging protocol that facilitates camera-based deep learning prediction and guidance for medical imaging protocols in accordance with one or more embodiments described herein.

FIGS. 4-5 illustrate example, non-limiting block diagrams showing how a prescribed imaging protocol can be determined from a prescription document in accordance with one or more embodiments described herein.

FIG. 6 illustrates an example, non-limiting block diagram of a prescribed imaging protocol in accordance with one or more embodiments described herein.

FIG. 7 illustrates a block diagram of an example, non-limiting system including a second deep learning neural network and a preparation determination that facilitates camera-based deep learning prediction and guidance for medical imaging protocols in accordance with one or more embodiments described herein.

FIGS. 8-12 illustrate example, non-limiting block diagrams showing how a preparation determination can be obtained from an image or video in accordance with one or more embodiments described herein.

FIG. 13 illustrates a block diagram of an example, non-limiting system including one or more prepared actions and one or more guidance actions that facilitates camera-based deep learning prediction and guidance for medical imaging protocols in accordance with one or more embodiments described herein.

FIG. 14 illustrates a block diagram of an example, non-limiting system excluding a prescription document that facilitates camera-based deep learning prediction and guidance for medical imaging protocols in accordance with one or more embodiments described herein.

FIG. 15 illustrates an example, non-limiting block diagram showing how a prescribed imaging protocol can be determined based on an image or video in accordance with one or more embodiments described herein.

FIG. 16 illustrates a flow diagram of an example, non-limiting computer-implemented method that facilitates camera-based deep learning prediction and guidance for medical imaging protocols in accordance with one or more embodiments described herein.

FIG. 17 illustrates an example, non-limiting block diagram showing how various artificial intelligence models can be trained in accordance with one or more embodiments described herein.

FIG. 18 illustrates a flow diagram of an example, non-limiting computer-implemented method that facilitates camera-based deep learning prediction and guidance for medical imaging protocols in accordance with one or more embodiments described herein.

FIG. 19 illustrates a block diagram of an example, non-limiting operating environment in which one or more embodiments described herein can be facilitated.

FIG. 20 illustrates an example networking environment operable to execute various implementations described herein.

### DETAILED DESCRIPTION

The following detailed description is merely illustrative and is not intended to limit embodiments or application/uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

A medical imaging scanner (e.g., a computed tomography (CT) scanner) can perform a wide variety of imaging protocols (e.g., defined by different configurations of scanning parameters) on medical patients (e.g., humans, animals, or otherwise). Such protocol variety, when combined with the realities of clinical time limitations and widely varying technician experience levels, can lead to unacceptably high scanning error rates. Indeed, scanner operators or technologists in clinics or hospitals often are required to scan numerous medical patients in short periods of time using different or respective imaging protocols. Because of such time pressure, the likelihood of a scanner operator or technologist selecting or utilizing for a given patient an imaging protocol that does not match that which the given patient's referring clinician has prescribed can be increased. Additionally, many geographic locations are suffering shortages of experienced scanner operators or technologists and are thus relying more heavily on inexperienced scanner operators or technologists. Due to the immense operational complexity of medical imaging scanners, such inexperience can further exacerbate the likelihood of non-prescribed imaging protocols being mistakenly or erroneously used.

So, systems or techniques that can address one or more of these technical problems can be desirable.

Various embodiments described herein can address one or more of these technical problems. One or more embodiments described herein can include systems, computer-implemented methods, apparatus, or computer program products that can facilitate camera-based deep learning prediction and guidance for medical imaging protocols. In other words, various embodiments described herein can leverage the image-analysis capabilities of deep learning so as to reduce or eliminate protocol-selection errors of medical imaging scanners. In particular, various embodiments described herein can include utilizing a first deep learning model to infer or predict what imaging protocol has been prescribed for a given medical patient. In some instances, such inference can be based on textual data typed or written by the given medical patient's attending or referring clinician. Note that the prescribed imaging protocol can specify not only the specific configuration that the operational parameters of the medical imaging scanner should be set to, but also a specific body pose of the given medical patient or a specific on-body scanner coil position of the given medical patient (e.g., in terms of anatomy or laterality) that should be used in order for the prescribed imaging protocol to be properly performed. In various aspects, various embodiments described herein can involve utilizing a second deep learning neural network to infer or predict whether or not the given medical patient is currently or presently prepared for the prescribed imaging protocol. In various cases, such inference can be based on a live camera feed that depicts the given medical patient in or on the gantry, table, or bay of the medical imaging scanner. More specifically, the second deep learning model can infer whether or not the actual body pose or actual on-body scanner coil position of the given medical patient matches that specified in the prescribed imaging protocol. If so, various embodiments described herein can involve automatically performing, or automatically prompting an operator or technologist for permission to perform, the prescribed imaging protocol on the given medical patient. If not, various embodiments described herein can instead involve displaying body pose guidance or scanner coil position guidance to the operator or technologist (e.g., to show or explain which body pose or scanner coil position is required by the prescribed imaging protocol). Such guidance can take any suitable form, such as on-screen instructions or diagrams, or such as light beams or laser beams being shined onto the body of the given medical patient. Such embodiments can thus be considered as automatically assisting the operator or technologist in correctly performing scans of medical patients, which can be desirable. Indeed, such embodiments can increase scanning throughput of the operator or technologist (e.g., the operator or technologist can, due to being assisted by various embodiments described herein, not have to perform unnecessary rescans of any given patient and can thus be able to correctly scan more patients in less time than would otherwise be possible). Moreover, because such embodiments can automatically provide body pose guidance or scanner coil position guidance to the operator or technologist in real-time, such embodiments can reduce or eliminate the capture of bad, artifact-riddled, or otherwise unusable scanned images, which can help to improve follow-on or downstream diagnosis or prognosis and thus patient outcomes.

Various embodiments described herein can be considered as a computerized tool (e.g., any suitable combination of computer-executable hardware or computer-executable software) that can facilitate camera-based deep learning prediction and guidance for medical imaging protocols. In various aspects, such computerized tool can comprise an access component, a protocol component, a preparation component, or a guidance component.

In various embodiments, there can be a medical imaging scanner. In various aspects, the medical imaging scanner can be any suitable medical modality, equipment, or device that can capture or generate medical scanned images (e.g., CT scanned images, X-ray scanned images, magnetic resonance imaging (MRI) scanned images) of any suitable medical patient.

In various embodiments, the medical imaging scanner can be associated with a preparatory camera. In various instances, the preparatory camera can be any suitable type of camera that can capture any suitable visible spectrum images or videos of the medical patient. In some cases, the preparatory camera can be in the same room as (e.g., physically built or integrated into) the medical imaging scanner, such that the preparatory camera can view the medical patient physically occupying a gantry, table, or bay of the medical imaging scanner. In other cases, the preparatory camera can be in a different room than the medical imaging scanner (e.g., can be located in an adjacent room in which the medical patient dons or doffs gear in preparation for their upcoming scan). In any case, the preparatory camera can capture real-time, or otherwise recent, images or videos of the medical patient as the medical patient prepares or waits for commencement of their scan.

In various instances, it can be desired to provide automated scanning assistance with respect to the medical imaging scanner and the medical patient. As described herein, the computerized tool can provide such automated assistance.

In various embodiments, the access component of the computerized tool can electronically access the medical imaging scanner or the preparatory camera. For instance, the access component can electronically interface or communicate with (e.g., send electronic commands to, read electronic signals from) the medical imaging scanner or the preparatory camera. In any case, the access component can be considered as a conduit through which other components of the computerized tool can electronically interact with (e.g., manipulate, execute, activate, deactivate, modify) the medical imaging scanner or the preparatory camera.

Furthermore, in various aspects, the access component can electronically access a prescription document or a preparation image or video. That is, the access component can electronically receive, retrieve, or otherwise obtain the prescription document or the preparation image or video, such that other components of the computerized tool can electronically interact with (e.g., read, write, edit, copy, manipulate) the prescription document or the preparation image or video. In various instances, the prescription document can be any suitable natural language or plain text sentences or sentence fragments that substantively or semantically convey clinical findings or observations regarding the medical patient, including a request, command, or prescription that the medical patient be scanned using the medical imaging scanner. Note that the access component can obtain the prescription document from any suitable electronic source (e.g., the prescription document can have been typed by or otherwise on behalf of a referring or attending physician of the medical patient; the prescription document can have been uploaded to a radiology information system (RIS) associated with the medical imaging scanner; and the access component can retrieve the prescription document from the RIS). In various cases, the preparation image or video can be one or more arrays of pixels or voxels that visually depict or illustrate the medical patient as they are preparing or waiting for commencement of an upcoming scan. Accordingly, the preparation image or video can be considered as being whatever visual data that the preparatory camera captures or records with respect to the medical patient. Note that the access component can obtain the preparation image or video from the preparatory camera.

In various embodiments, the protocol component can electronically store, maintain, control, or otherwise access a first deep learning neural network. In various aspects, the first deep learning neural network can exhibit any suitable deep learning internal architecture. For example, the first deep learning neural network can include any suitable numbers of any suitable types of layers (e.g., input layer, one or more hidden layers, output layer, any of which can be convolutional layers, dense layers, long short-term memory (LSTM) layers, transformer layers, non-linearity layers, pooling layers, batch normalization layers, or padding layers). As another example, the first deep learning neural network can include any suitable numbers of neurons in various layers (e.g., different layers can have the same or different numbers of neurons as each other). As yet another example, the first deep learning neural network can include any suitable activation functions (e.g., softmax, sigmoid, hyperbolic tangent, rectified linear unit) in various neurons (e.g., different neurons can have the same or different activation functions as each other). As still another example, the first deep learning neural network can include any suitable interneuron connections or interlayer connections (e.g., forward connections, skip connections, recurrent connections).

Regardless of its internal architecture, the first deep learning neural network can be configured to receive as input any suitable text and to determine as output an imaging protocol that is specified, recited, called for, or otherwise prescribed by such inputted text. Accordingly, the protocol component 116 can electronically execute the first deep learning neural network on the prescription document, thereby causing the first deep learning neural network to determine which specific imaging protocol is prescribed by the prescription document.

In some cases, the first deep learning neural network can be structured as a text classifier. So, the protocol component can electronically execute the first deep learning neural network on the prescription document, and such execution can yield a protocol classification label. More specifically, the protocol component can feed the prescription document to the input layer of the first deep learning neural network, the prescription document can complete a forward pass through the one or more hidden layers of the first deep learning neural network, and the output layer of the first deep learning neural network can calculate the protocol classification label based on activations provided by the one or more hidden layers of the first deep learning neural network. In various aspects, the protocol classification label can indicate an imaging protocol of the medical imaging scanner that the first deep learning neural network believes or infers is requested or called-for by the prescription document. In particular, there can be a plurality of defined imaging protocols that the medical imaging scanner can possibly implement (e.g., different protocols can specify different radiation levels or different gantry speeds to be used for scanning different body parts), and the protocol classification label can indicate which one of the plurality of defined imaging protocols that the prescription document (in the opinion of the first deep learning neural network) recites or prescribes for the medical patient.

In other cases, the first deep learning neural network can instead be structured as a large language model (e.g., ChatGPT). In such situations, the protocol component can execute the first deep learning neural network on the prescription document and on a protocol identification prompt, and such execution can yield a protocol indication. More specifically, the protocol identification prompt can be unstructured or plain text that asks or commands identification, description, or explanation of whatever imaging protocol is indicated or specified in the prescription document. In various aspects, the protocol component can concatenate the prescription document and the protocol identification prompt together. In various instances, the protocol component can feed that concatenation to the input layer of the first deep learning neural network, that concatenation can complete a forward pass through the one or more hidden layers of the first deep learning neural network, and the output layer of the first deep learning neural network can calculate the protocol indication based on activations provided by the one or more hidden layers of the first deep learning neural network. In various cases, the protocol indication can be synthesized text that is based on the prescription document, and that substantively or semantically responds to the protocol identification prompt. In other words, the protocol indication can be unstructured or plain text that names, states, describes, or explains whatever specific imaging protocol that (in the opinion of the first deep learning neural network) the prescription document requests or calls for.

In any case, the protocol component can leverage the first deep learning neural network so as to identify the specific imaging protocol that the prescription document prescribes for the medical patient. Such imaging protocol can be referred to as the prescribed imaging protocol. In various aspects, the prescribed imaging protocol can be associated with or otherwise defined by a requisite scanning parameter configuration. In other words, the medical imaging scanner can have any suitable scanning parameters (e.g., radiation level, gantry speed, field of view, matrix size), and the requisite scanning parameter configuration can be whatever specific combination of values or states that those scanning parameters should be set to so as to perform or accomplish the prescribed imaging protocol. However, in addition to the requisite scanning parameter configuration, the prescribed imaging protocol can be associated with or otherwise defined by a requisite body pose or a requisite scanner coil position. Indeed, in various instances, the prescribed imaging protocol can be intended or designed to be applied to patients whose bodies are physically oriented with respect to the medical imaging scanner in some specific fashion (e.g., prone, supine, side-leaning, head-first, feet-first), and the requisite body pose can be or refer to that specific orientation. Likewise, in various cases, the prescribed imaging protocol can be intended or designed to be applied to patients that are wearing scanner coils at a particular anatomical location (e.g., coil on left leg, coil on right arm, coil on head), and the requisite scanner coil position can be or refer to that specific anatomical location.

In various embodiments, the preparation component can electronically store, maintain, control, or otherwise access a second deep learning neural network. In various aspects, the second deep learning neural network can exhibit any suitable deep learning internal architecture. For example, the second deep learning neural network can include any suitable numbers of any suitable types of layers (e.g., input layer, one or more hidden layers, output layer, any of which can be convolutional layers, dense layers, LSTM layers, transformer layers, non-linearity layers, pooling layers, batch normalization layers, or padding layers). As another example, the second deep learning neural network can include any suitable numbers of neurons in various layers (e.g., different layers can have the same or different numbers of neurons as each other). As yet another example, the second deep learning neural network can include any suitable activation functions (e.g., softmax, sigmoid, hyperbolic tangent, rectified linear unit) in various neurons (e.g., different neurons can have the same or different activation functions as each other). As still another example, the second deep learning neural network can include any suitable interneuron connections or interlayer connections (e.g., forward connections, skip connections, recurrent connections).

Regardless of its internal architecture, the second deep learning neural network can be configured as a computer vision model. That is, the second deep learning neural network can be configured to receive as input any suitable image or video data and to localize as output certain objects of interest in such inputted image or video data. In some aspects, such objects of interest can include various body parts (e.g., head, right eye, left eye, right arm, left arm). In some instances, such objects of interest can include wearable scanner coils. Accordingly, the preparation component 118 can electronically execute the second deep learning neural network on the preparation image or video, thereby causing the second deep learning neural network to produce a set of body part localizations or a scanner coil localization. More specifically, the preparation component can feed the preparation image or video to the input layer of the second deep learning neural network, the preparation image or video can complete a forward pass through the one or more hidden layers of the second deep learning neural network, and the output layer of the second deep learning neural network can calculate the set of body part localizations or the scanner coil localization based on activations provided by the one or more hidden layers of the second deep learning neural network.

In various aspects, the set of body part localizations can include any suitable number of localizations respectively corresponding to any suitable number of distinct body parts of the medical patient. In various instances, each body part localization can be any suitable electronic data that indicates (in the opinion of the second deep learning neural network) an intra-image or intra-video location of a respective body part of the medical patient (e.g., can be landmark coordinates of the respective body part; can be a bounding box circumscribing the respective body part; can be a segmentation mask covering the respective body part). Similarly, the scanner coil localization can be any suitable electronic data that indicates (e.g., in the opinion of the second deep learning neural network) an intra-image or intra-video location of a scanner coil worn by the medical patient (e.g., can be landmark coordinates of the scanner coil; can be a bounding box circumscribing the scanner coil; can be a segmentation mask covering the scanner coil).

In various aspects, the preparation component can electronically determine whether or not the medical patient is properly prepared for the prescribed imaging protocol, by comparing: the set of body part localizations or the scanner coil localization; to the requisite body pose or the requisite scanner coil position. Indeed, in situations where the preparatory camera is viewing or aimed at the gantry, table, or bay of the medical imaging scanner, the set of body part localizations can be considered as collectively indicating or conveying a current or present body pose of the medical patient as the medical patient waits on or in the gantry, table, or bay (e.g., head being located above feet can indicate head-first pose; head being located below feet can indicate feet-first pose). Furthermore, the set of body part localizations and the scanner coil localization together can be considered as collectively indicating a current or present scanner coil position of the medical patient (e.g., scanner coil localization coinciding with right arm localization can indicate that the scanner coil is worn on the right arm of the medical patient). If the current or present body pose does not match the requisite body pose, the preparation component can conclude or determine that the medical patient is not prepared for the prescribed imaging protocol. Likewise, if the current or present scanner coil position does not match the requisite scanner coil position, the preparation component can conclude or determine that the medical patient is not prepared for the prescribed imaging protocol. However, if the current or present body pose matches the requisite body pose, and if the current or present scanner coil position matches the requisite scanner coil position, the preparation component can instead conclude or determine that the medical patient is prepared for the prescribed imaging protocol.

In various embodiments, the guidance component can initiate or perform any suitable electronic actions, based on the determination or conclusion of the preparation component. For instance, if the preparation component determines or concludes that the medical patient is prepared for the prescribed imaging protocol, then the guidance component can initiate or perform any suitable prepared actions. Such prepared actions can include: instructing or commanding the medical imaging scanner to begin the prescribed imaging protocol; or prompting, via a graphical user-interface (GUI) of the medical imaging scanner, an operator of the medical imaging scanner for permission to begin the prescribed imaging protocol. On the other hand, if the preparation component determines or concludes that the medical patient is not yet prepared for the prescribed imaging protocol, then the guidance component can initiate or perform any suitable guidance actions. If the preparation component determined that the medical patient was unprepared due to failure to satisfy the requisite body pose, such guidance actions can include rendering, on the GUI, a notification indicating that the medical patient will not be prepared until the requisite body pose is achieved. Similarly, if the preparation component determined that the medical patient was unprepared due to failure to satisfy the requisite scanner coil position, such guidance actions can include rendering, on the GUI, a notification indicating that the medical patient will not be prepared until the requisite scanner coil position is achieved. In some aspects, there can be an actuatable or movable light or laser associated with the medical imaging scanner, where such light or laser can be controllably pointed or aimed at the body of the medical patient. In such situations, if the preparation component determined that the medical patient was unprepared due to failure to satisfy the requisite scanner coil position, the guidance actions can include causing the light or laser to shine a visible beam onto the body of the medical patient, such that the visible beam lands at or otherwise visually indicates the requisite scanner coil position (e.g., lands at or visually indicates whatever specific portion of the medical patient's body that the scanner coil should be moved to). Accordingly, if the medical patient is not yet prepared for the prescribed imaging protocol, the guidance component can be considered as helping or assisting the operator of the medical imaging scanner to quickly or efficiently make the medical patient prepared (e.g., by indicating how the patient's body pose should be changed or how the patient's scanner coil position should be changed).

Note that, in order for the prescribed imaging protocol to be correctly or accurately identified, or for the preparedness determination to be correctly or accurately made, the herein-described machine learning models (e.g., the first and second deep learning neural networks) should first undergo training. In various cases, the computerized tool can train such machine learning models using any suitable training paradigms (e.g., via supervised training, unsupervised training, or reinforcement learning).

Various embodiments described herein can be employed to use hardware or software to solve problems that are highly technical in nature (e.g., to facilitate camera-based deep learning prediction and guidance for medical imaging protocols), that are not abstract and that cannot be performed as a set of mental acts by a human. Further, some of the processes performed can be performed by a specialized computer (e.g., text classifiers, LLMs, computer vision models) for carrying out defined acts related to medical imaging scanners.

For example, such defined acts can include: inferring, by a device operatively coupled to a processor and via execution of a first deep learning neural network (e.g., text classifier or LLM executed on a prescription document), a prescribed imaging protocol that is to be performed by a medical imaging scanner on a medical patient; inferring, by the device and via execution of a second deep learning neural network (e.g., computer vision model) on a preparation image or video of the medical patient that is captured by a camera associated with the medical imaging scanner, whether or not the medical patient is prepared for the prescribed imaging protocol; and initiating, by the device and in response to an inference that the medical patient is not prepared for the prescribed imaging protocol, an electronic guidance action that explains or shows how to make the medical patient prepared for the prescribed imaging protocol. In various instances, such defined acts can further include: rendering, by the device, in response to an inference that the medical patient is prepared for the prescribed imaging protocol, and on a graphical user-interface of the medical imaging scanner, a notification indicating that the prescribed imaging protocol is ready to be performed and requesting a user of the medical imaging scanner to approve performance of the prescribed imaging protocol; or instructing, by the device, the medical imaging scanner to perform the prescribed imaging protocol. In various cases, the second deep learning neural network can receive as input the preparation image or video and produce as output a localization indicating a current body pose or orientation of the medical patient, and the device can infer that the medical patient is not prepared, in response to the current body pose or orientation not matching a requisite body pose or orientation specified in the prescribed imaging protocol. In various aspects, the second deep learning neural network can receive as input the preparation image or video and produce as output a localization indicating a current scanner coil position on the medical patient, and the device can infer that the medical patient is not prepared, in response to the current scanner coil position not matching a requisite scanner coil position specified in the prescribed imaging protocol. In various instances, the current scanner coil location of the medical patient inferred by the second deep learning neural network can fail to match the requisite scanner coil position specified in the prescribed imaging protocol, and the electronic guidance action can comprise shining a light or laser associated with the medical imaging scanner onto the body of the medical patient in accordance with the requisite scanner coil position.

Such defined acts are not performed manually by humans. Indeed, neither the human mind nor a human with pen and paper can: electronically execute a text classifier or LLM on a prescription document, thereby identifying an imaging protocol that is to be performed on a given patient by a medical imaging scanner; electronically capture live or real-time image or video that depicts the given patient preparing or waiting for commencement of the imaging protocol; electronically execute a computer vision model on that image or video, thereby producing body part localizations or wearable scanner coil localizations for the medical patient; electronically compare those localizations to a required body pose or a required scanner coil position specified by or otherwise associated with the imaging protocol, so as to determine whether the given patient is prepared for the imaging protocol; and electronically provide body pose or scanner coil guidance (e.g., via GUI displays, or via real-world light or laser beams) in response to determining that the given patient is not yet prepared for the imaging protocol. Indeed, medical imaging scanners (e.g., MRI scanners, CT scanners, X-ray scanners) are inherently-computerized, hardware-based constructs that simply cannot be meaningfully implemented in any way by the human mind without computers. Additionally, deep learning neural networks (e.g., text classifiers, LLMs, computer vision models) are inherently computerized, software-based constructs that cannot be meaningfully trained or executed in any way by the human mind without computers. Accordingly, a computerized tool that leverages the text-analysis or image-analysis capabilities of deep learning neural networks so as to automatically identify a medical imaging protocol that is to be applied to a patient, so as to automatically check whether or not a body pose or wearable scanner coil position of the patient is consistent with those required by the medical imaging protocol, and so as to rectify any identified inconsistencies with on-screen instruction or with on-body light beams is likewise inherently-computerized and cannot be implemented in any sensible, practical, or reasonable way without computers.

Moreover, various embodiments described herein can integrate into a practical application various teachings relating to the field of medical imaging scanners. As described above, a medical imaging scanner can perform a myriad of possible imaging protocols. Also as described above, different imaging protocols can be prescribed for different medical patients. Because of time limitations, long working hours, and widely varying experience levels, a medical imaging technologist can have an increased likelihood of mistakenly or erroneously performing medical imaging scans (e.g., can accidently select or load a wrong, incorrect, or not-prescribed protocol for a given patient; can select or load the correct or prescribed protocol for a given patient, but can accidently fail to ensure that the patient's body pose or wearable scanner coil position are consistent with the correct or prescribed protocol).

Various embodiments described herein can address one or more of these technical problems. In particular, when given a prescription document (e.g., clinical notes written by a referring physician and stored on a clinical RIS database) of a specific patient that is about to undergo a medical scan, various embodiments described herein can execute a first deep learning neural network (e.g., text classifier, LLM) on the prescription document, thereby identifying a specific imaging protocol that has been prescribed for the specific patient. Moreover, various embodiments described herein can capture live images or video of the specific patient as they are preparing or otherwise waiting for their scan (e.g., preparing or waiting in or on the gantry, table, or bay of a medical imaging scanner; preparing or waiting in a donning or doffing room associated with the medical imaging scanner). In various aspects, various embodiments described herein can execute a second deep learning neural network (e.g., computer vision model) on the live images or video, thereby identifying a current body pose or a current wearable scanner coil position of the specific patient. In various instances, various embodiments described herein can determine whether the current body pose or current wearable scanner coil position are consistent with those that are listed or specified as required or necessary for the specific imaging protocol. If they are consistent, various embodiments can automatically begin the specific imaging protocol, or can display a GUI message to an operator that asks for permission to begin the specific imaging protocol. On the other hand, if they are inconsistent, various embodiments described herein can provide real-time guidance to the operator (e.g., a GUI message indicating that the body pose or wearable scanner coil position of the specific patient must be corrected; shining visible lights onto the body of the specific patient, so as to indicate where the wearable scanner coil should be located on the specific patient). Such embodiments can be considered as automatically helping or assisting the operator so as to reduce or avoid erroneous or flawed scans, thereby saving time and effort for the operator, thereby saving time and effort for patients (e.g., eliminates or reduces need for repeat scans), and thereby improving clinical outcomes (e.g., eliminates or reduces generation of inaccurate, flawed, or artifact-ridden medical images). Therefore, various embodiments described herein can be considered as a clever or inventive technique or pipeline that leverages camera-based deep learning to assist in the performance of medical imaging scans on medical patients. Thus, various embodiments described herein certainly constitute a tangible and concrete technical improvement or technical advantage in the field of medical imaging scanners. Accordingly, such embodiments clearly qualify as useful and practical applications of computers.

Furthermore, various embodiments described herein can control real-world tangible devices based on the disclosed teachings. For example, various embodiments described herein can instruct or cause real-world medical imaging scanners (e.g., CT scanner, MRI scanner) to perform real-world scans on real-world patients. Moreover, various embodiments described herein can cause real-world illumination devices to shine visible light beams or visible laser beams onto the bodies of such real-world patients.

It should be appreciated that the herein figures and description provide non-limiting examples of various embodiments and are not necessarily drawn to scale.

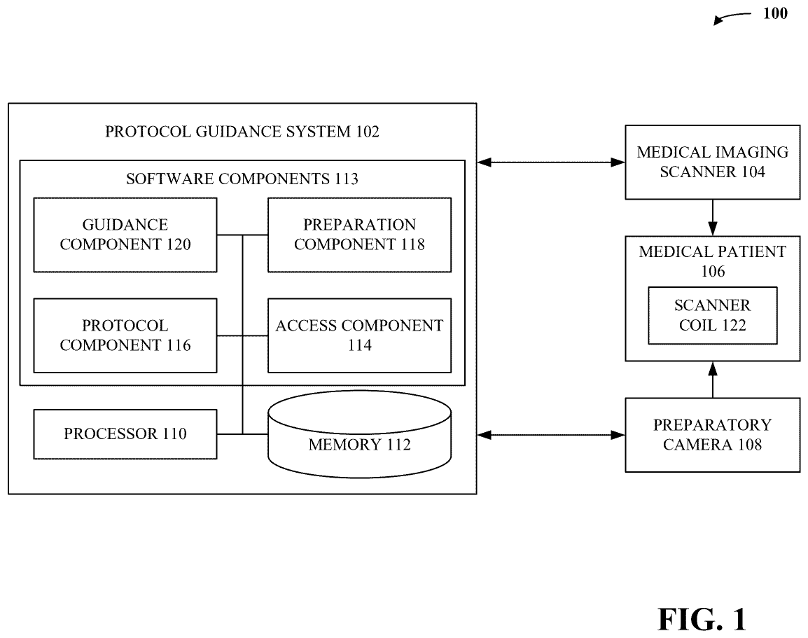

FIG. 1 illustrates a block diagram of an example, non-limiting system 100 that can facilitate camera-based deep learning prediction and guidance for medical imaging protocols in accordance with one or more embodiments described herein. As shown, a protocol guidance system 102 can be electronically integrated with a medical imaging scanner 104 or a preparatory camera 108.

In various embodiments, the medical imaging scanner 104 can be any suitable medical image-capture modality or equipment that can capture or otherwise generate (e.g., via X-ray emission or electromagnetism) medical images. As a non-limiting example, the medical imaging scanner 104 can be an X-ray scanner that is configured to capture or generate X-ray scanned images of any suitable anatomical structures (e.g., organs, tissues, bodily cavities, bodily fluids) of a medical patient 106. As another non-limiting example, the medical imaging scanner 104 can be a CT scanner that is configured to capture or generate CT scanned images of any suitable anatomical structures of the medical patient 106. As even another non-limiting example, the medical imaging scanner 104 can be a positron emission tomography (PET) scanner that is configured to capture or generate PET scanned images of any suitable anatomical structures of the medical patient 106. As yet another non-limiting example, the medical imaging scanner 104 can be a nuclear medicine (NM) scanner that is configured to capture or generate NM scanned images of any suitable anatomical structures of the medical patient 106. As still another non-limiting example, the medical imaging scanner 104 can be an ultrasound scanner that is configured to capture or generate ultrasound scanned images of any suitable anatomical structures of the medical patient 106. As another non-limiting example, the medical imaging scanner 104 can be an MRI scanner that is configured to capture or generate MRI scanned images of any suitable anatomical structures of the medical patient 106.

Although not explicitly shown in the figures, the medical imaging scanner 104 can be electronically integrated with any suitable human-computer interface device, which can be remote from or local to the medical imaging scanner 104. Accordingly, an operator or user associated with the medical imaging scanner 104 can interact with or otherwise control the medical imaging scanner 104. Some non-limiting examples of the human-computer interface device can be a keyboard of the medical imaging scanner 104, a keypad of the medical imaging scanner 104, a touchscreen of the medical imaging scanner 104, or a voice-command system of the medical imaging scanner 104.

Although not explicitly shown in the figures, the medical imaging scanner 104 can have or otherwise be associated with any suitable number of any suitable types of scanning parameters. In various aspects, a scanning parameter can be any suitable configurable setting of the medical imaging scanner 104 that can be selectively controlled by the user or operator so as to commensurately control how the medical imaging scanner 104 operates, functions, or otherwise performs scans. As some non-limiting examples, a scanning parameter can be any of the following: a sequence-type parameter of the medical imaging scanner 104 (e.g., possible or selectable values or states of such parameter can include a T1-weighted sequence type, a T2-weighted sequence type, a proton density sequence type, a diffusion-weighted sequence type, or a fluid-attenuated inversion recovery sequence type); a slice-thickness parameter of the medical imaging scanner 104 (e.g., possible or selectable values or states of such parameter can include 1 millimeter (mm) slice thickness, 3 mm slice thickness, or 10 mm slice thickness); a slice orientation parameter of the medical imaging scanner 104 (e.g., possible or selectable values or states of such parameter can include an axial slice orientation, a coronal slice orientation, or a sagittal slice orientation); a field of view (FOV) parameter of the medical imaging scanner 104 (e.g., possible or selectable values or states of such parameter can include a 100×100 mm² FOV, a 150×150 mm² FOV, a 200×200 mm² FOV, or a 400×400 mm² FOV); a matrix size parameter of the medical imaging scanner 104 (e.g., possible or selectable values or states of such parameter can include a 128-pixels-by-128-pixels matrix size, a 256-pixels-by-256-pixels matrix size, or 512-pixels-by-512-pixels matrix size); a repetition time (TR) parameter of the medical imaging scanner 104 (e.g., possible or selectable values or states of such parameter can include a 2 millisecond (ms) TR, a 500 ms TR, a 2000 ms TR, or a 5000 ms TR); an echo time (TE) parameter of the medical imaging scanner 104 (e.g., possible or selectable values or states of such parameter can include a 2 millisecond (ms) TE, a 20 ms TE, an 80 ms TE, or a 200 ms TE); or a number of excitations (NEX) parameter of the medical imaging scanner 104 (e.g., possible or selectable values or states of such parameter can include 1 NEX (meaning that a resulting image is formed from a single scanning excitation), 2 NEX (meaning that a resulting image is the average of two scanning excitations), or 3 NEX (meaning that a resulting image is the average of three scanning excitations).

In various aspects, a scanner coil 122 can be associated with the medical imaging scanner 104. In various instances, the scanner coil 122 can be any suitable wearable device or equipment that aids or otherwise assists in any suitable fashion the medical imaging scanner 104 to pass electromagnetic radiation into or through the body of the medical patient 106. In some cases, the scanner coil 122 can alternatively be referred to as a surface coil. In any case, the scanner coil 122 can be physically worn by the medical patient 106. In other words, the scanner coil 122 can be physically situated on, physically wrapped around, or otherwise fully or partially physically enclosing or encompassing some external body part of the medical patient 106. As some non-limiting examples, the scanner coil 122 can be worn on: a head of the medical patient 106; a right or left shoulder of the medical patient 106; a right or left upper arm of the medical patient 106; a right or left elbow of the medical patient 106; a right or left forearm of the medical patient 106; a right or left wrist or hand of the medical patient 106; a torso of the medical patient 106; a right or left hip of the medical patient 106; a right or left thigh of the medical patient 106; a right or left knee of the medical patient 106; a right or left calf of the medical patient 106; or a right or left ankle or foot of the medical patient 106.

In various embodiments, the preparatory camera 108 can be any suitable image-capture device that can view the medical patient 106 as the medical patient 106 prepares for, gets ready for, or otherwise waits for the medical imaging scanner 104 to scan them. In various aspects, the preparatory camera 108 can be physically integrated or otherwise built into the medical imaging scanner 104. Accordingly, the preparatory camera 108 can be able to view the medical patient 106 as they wear the scanner coil 122 and sit on or in, lie on or in, or otherwise physically occupy an actuatable table of, a gantry of, or an imaging bay of the medical imaging scanner 104. However, this is a mere non-limiting example. In other aspects, the preparatory camera 108 can be physically remote or separate from the medical imaging scanner 104, but can nevertheless be in the same room as the medical imaging scanner 104. In such cases, the preparatory camera 108 can, notwithstanding being physically remote or separate from the medical imaging scanner 104, still be able to view the medical patient 106 as they wear the scanner coil 122 and sit on or in, lie on or in, or otherwise occupy the table, gantry, or imaging bay of the medical imaging scanner 104. In even other aspects, the preparatory camera 108 can be in a separate or different room than the medical imaging scanner 104. Indeed, high-traffic hospitals can have a first room that houses the medical imaging scanner 104 and an adjacent or otherwise nearby second room in which patients who are queueing for a scan don the scanner coil 122. In such situations, the preparatory camera 108 can be located in the second room rather than the first room, such that the preparatory camera 108 cannot view the medical patient 106 sitting on or in, lying on or in, or occupying the table, gantry, or bay of the medical imaging scanner 104, but such that the preparatory camera 108 can nevertheless view the medical patient 106 wearing the scanner coil 122.

It should be appreciated that the preparatory camera 108 can exhibit any suitable architecture or construction. For instance, the preparatory camera 108 can comprise or otherwise be made up of any suitable types of optical lens, any suitable types of shutters, or any suitable types of photodetection mechanisms. In some aspects, the preparatory camera 108 can capture images or videos of the medical patient 106 in the visible spectrum. In other aspects, the preparatory camera 108 can capture images or videos of the medical patient 106 in any suitable non-visible spectrum, such as infrared images or videos, or such as thermal images or videos.

In various cases, it can be desired to automatically assist the operator or user of the medical imaging scanner 104 in performing a scan on the medical patient 106. As described herein, the protocol guidance system 102 can facilitate such automated assistance.

In various embodiments, the protocol guidance system 102 can comprise a processor 110 (e.g., computer processing unit, microprocessor) and a non-transitory computer-readable memory 112 that is operably or operatively or communicatively connected or coupled to the processor 110. The non-transitory computer-readable memory 112 can store computer-executable instructions which, upon execution by the processor 110, can cause the processor 110 or other components of the protocol guidance system 102 (e.g., access component 114, protocol component 116, preparation component 118, guidance component 120) to perform one or more acts. In various embodiments, the non-transitory computer-readable memory 112 can store computer-executable components (e.g., access component 114, protocol component 116, preparation component 118, guidance component 120), and the processor 110 can execute the computer-executable components.

In various embodiments, the protocol guidance system 102 can comprise an access component 114. In various aspects, the access component 114 can electronically access or otherwise electronically communicate in any suitable fashion with the medical imaging scanner 104 or with the preparatory camera 108. For instance, the access component 114 can electronically transmit any suitable electronic data to, or receive any suitable electronic data from, the medical imaging scanner 104 or the preparatory camera 108. Accordingly, the access component 114 can be considered as a proxy or conduit by which other components of the protocol guidance system 102 can electronically interact with the medical imaging scanner 104 or with the preparatory camera 108.

In various aspects, the access component 114 can, as described herein, further electronically access a prescription document and a preparation image or video associated with the medical patient 106.

In various embodiments, the protocol guidance system 102 can comprise a protocol component 116. In various aspects, the protocol component 116 can, as described herein, electronically identify, via execution of a first deep learning neural network on the prescription document, a prescribed imaging protocol that is to be performed on the medical patient 106.

In various embodiments, the protocol guidance system 102 can comprise a preparation component 118. In various instances, the preparation component 118 can, as described herein, electronically determine, via execution of a second deep learning neural network on the preparation image or video, whether or not the medical patient 106 is properly prepared for performance of the prescribed imaging protocol.

In various embodiments, the protocol guidance system 102 can comprise a guidance component 120. In various cases, the guidance component 120 can, as described herein, electronically initiate any suitable actions based on the preparation determination of the preparation component 118 (e.g., initiate the prescribed imaging protocol if the medical patient is already prepared; provide electronic corrective guidance to the user or operator of the medical imaging scanner 104 if the medical patient 106 is not yet prepared).

Note that, in various instances, the access component 114, the protocol component 116, the preparation component 118, and the guidance component 120 can collectively be considered as being one or more software components 113 of the protocol guidance system 102. In various aspects, it should be appreciated that the one or more software components 113 are described primarily herein as comprising four components (e.g., the access component 114, the protocol component 116, the preparation component 118, and the guidance component 120) for ease of explanation and illustration. However, the one or more software components 113 are not limited to being implemented as exactly such four components in every embodiment. Indeed, in some embodiments, the functionalities described herein of such four components can be combined in any suitable fashions, so as to be implemented in or by fewer than four components (e.g., in some cases, a single component can perform all of the functionalities that are described herein with respect to the access component 114, the protocol component 116, the preparation component 118, and the guidance component 120). In other embodiments, the functionalities described herein of such four components can instead be distributed, separated, split, or fragmented in any suitable fashions, so as to be implemented in or by more than four components (e.g., two or more components can facilitate the functionalities that are performable by the access component 114; two or more components can facilitate the functionalities that are performable by the protocol component 116; two or more components can facilitate the functionalities that are performable by the preparation component 118; two or more components can facilitate the functionalities that are performable by the guidance component 120).

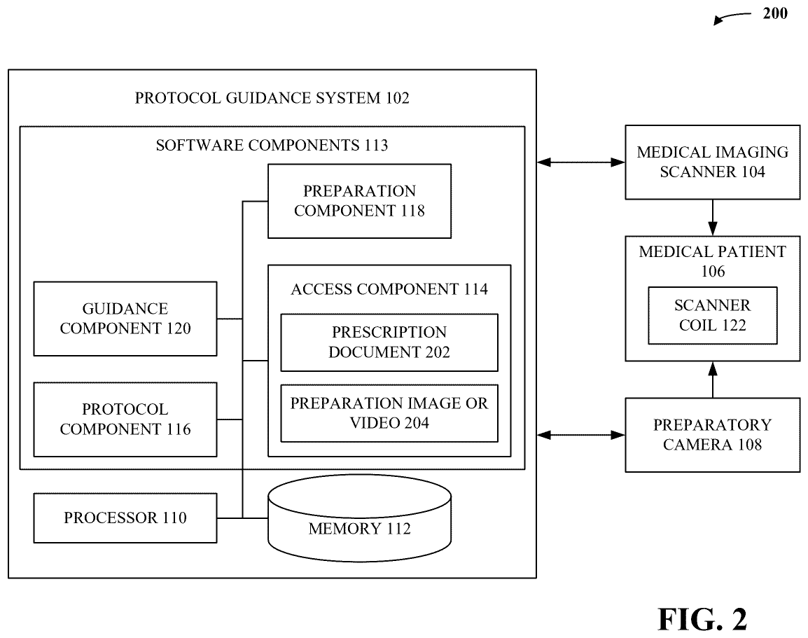

FIG. 2 illustrates a block diagram of an example, non-limiting system 200 including a prescription document and a preparation image or video that can facilitate camera-based deep learning prediction and guidance for medical imaging protocols in accordance with one or more embodiments described herein. As shown, the system 200 can, in some cases, comprise the same components as the system 100, and can further comprise a prescription document 202 and a preparation image or video 204.

In various embodiments, the access component 114 can electronically access the prescription document 202 from any suitable electronic source. As a non-limiting example, the access component 114 can electronically receive, electronically retrieve, or otherwise electronically obtain the prescription document 202 from an RIS database that is associated with the medical imaging scanner 104 (e.g., the RIS database and the medical imaging scanner 104 can belong to the same clinic or hospital). In any case, the prescription document 202 can be or otherwise comprise one or more unstructured or plain text declarative sentences or sentence fragments that semantically explain, describe, name, indicate, or otherwise convey any suitable imaging protocol of the medical imaging scanner 104 that a referring or attending physician or other medical professional has requested, called for, or otherwise prescribed for the medical patient 106. In various aspects, the prescription document 202 can be electronically typed or otherwise created (e.g., via voice-to-text transcription) by or on behalf of the referring or attending physical or other medical professional. In various instances, the prescription document 202 can utilize idiosyncratic or otherwise non-standardized language or word-choice to explain, describe, name, indicate, or otherwise convey the imaging protocol for the medical patient 106. As a non-limiting example, there can be various different ways of textually prescribing a scan for the right knee of the medical patient 106, such as “scan right knee”, such as “rt knee”, or such as “knee R”.

In various embodiments, the access component 114 can electronically access the preparation image or video 204 from any suitable electronic source. As a non-limiting example, the access component 114 can electronically receive, electronically retrieve, or otherwise electronically obtain the preparation image or video 204 from the preparatory camera 108. However, this is a mere non-limiting example. In other aspects, the preparatory camera 108 can cause the preparation image or video 204 to be stored or maintained in any suitable electronically-accessible database, and the access component 114 can electronically receive, electronically retrieve, or otherwise electronically obtain the preparation image or video 204 from that database. In any case, the preparation image or video 204 can be electronically captured or otherwise recorded by the preparatory camera 108. Accordingly, the preparation image or video 204 can be one or more (e.g., a time series of) x-by-y arrays of pixels, for any suitable positive integers x and y, that visually illustrate or depict the medical patient 106 as they prepare or otherwise wait for their turn to be scanned by the medical imaging scanner 104. If the preparatory camera 108 is located in the same room as the medical imaging scanner 104, the preparation image or video 204 can visually illustrate the medical patient 106 wearing the scanner coil 122 and sitting on or in, lying on or in, or otherwise occupying the table, gantry, or bay of the medical imaging scanner 104. On the other hand, if the preparatory camera 108 is instead located in a separate room than the medical imaging scanner 104, the preparation image or video 204 can visually illustrate the medical patient 106 wearing the scanner coil 122 but not sitting on or in, lying on or in, or otherwise occupying the table, gantry, or bay of the medical imaging scanner 104.

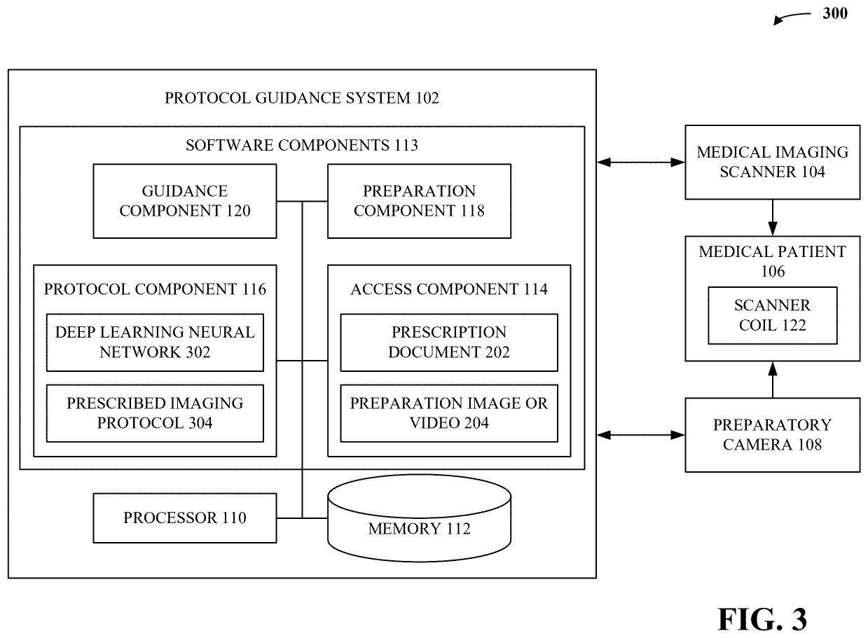

FIG. 3 illustrates a block diagram of an example, non-limiting system 300 including a first deep learning neural network and a prescribed imaging protocol that can facilitate camera-based deep learning prediction and guidance for medical imaging protocols in accordance with one or more embodiments described herein. As shown, the system 300 can, in some cases, comprise the same components as the system 200, and can further comprise a deep learning neural network 302 and a prescribed imaging protocol 304.

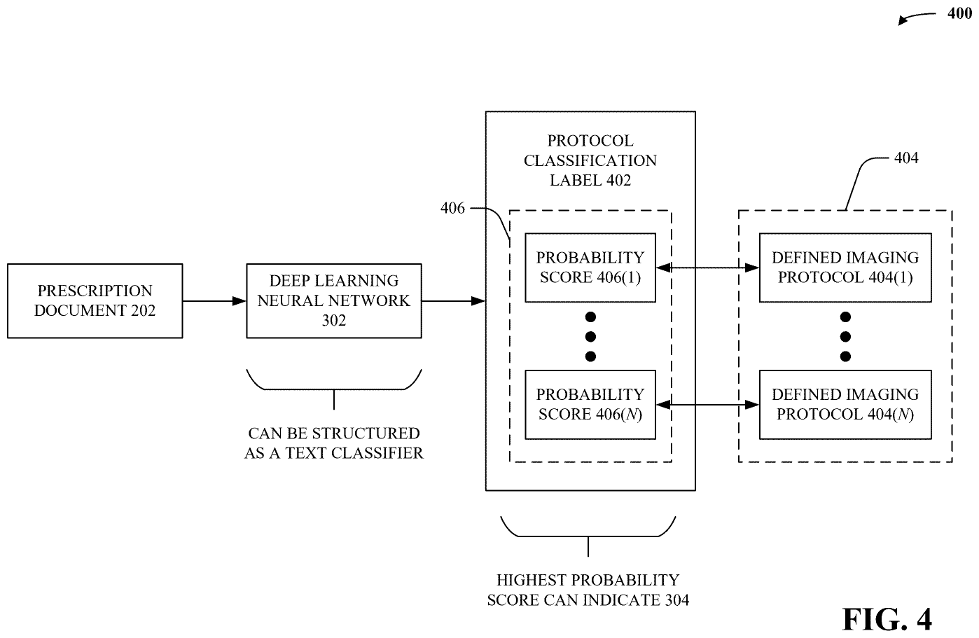

In various embodiments, the protocol component 116 can electronically store, electronically maintain, electronically control, or otherwise electronically access the deep learning neural network 302. In various aspects, as described herein, the protocol component 116 can electronically leverage the deep learning neural network 302 so as to identify the prescribed imaging protocol 304 based on the prescription document 202. Non-limiting aspects are described with respect to FIGS. 4-6.

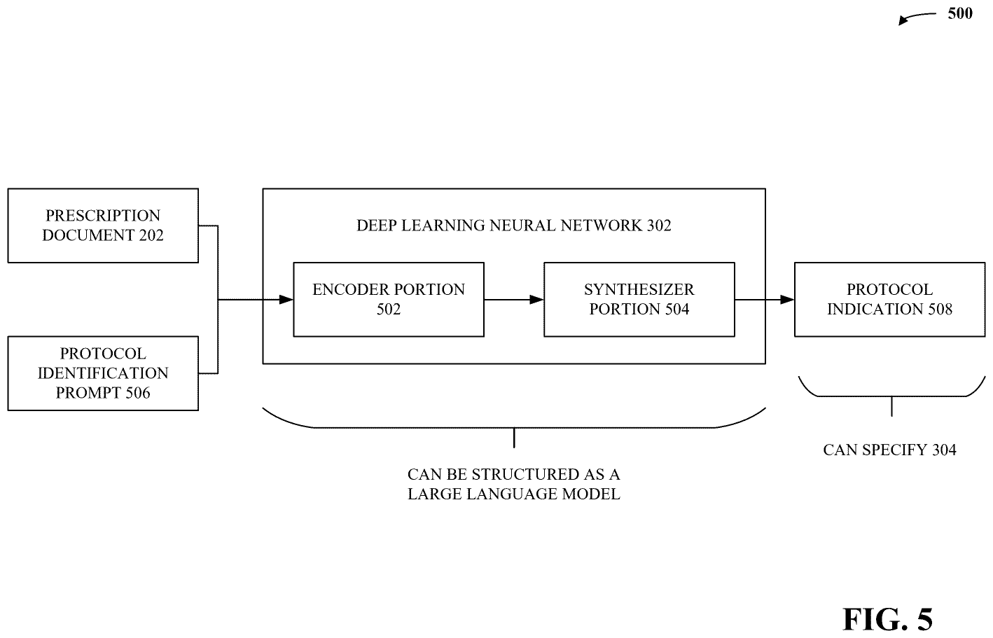

FIGS. 4-5 illustrate example, non-limiting block diagrams 400 and 500 showing how the prescribed imaging protocol 304 can be determined from the prescription document 202 in accordance with one or more embodiments described herein.

First, consider FIG. 4. In various embodiments, as shown, the deep learning neural network 302 can be configured as a text classifier having any suitable deep learning internal architecture. For instance, in various cases, the deep learning neural network 302 can have an input layer, one or more hidden layers, and an output layer. In various aspects, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. As even another example, any of such input layer, one or more hidden layers, or output layer can be LSTM layers, whose learnable or trainable parameters can be input-state weight matrices or hidden-state weight matrices. As yet another example, any of such input layer, one or more hidden layers, or output layer can be transformer layers, whose learnable or trainable parameters can be single-head or multi-head attention blocks or other weight matrices. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

Regardless of the specific internal architecture (e.g., of the specific numbers, types, or organizations of layers) that is implemented in the deep learning neural network 302, the deep learning neural network 302 can be configured to receive as input textual data and to produce as output a classification label for that inputted textual data. Accordingly, the protocol component 116 can electronically execute the deep learning neural network 302 on the prescription document 202, and such execution can cause the deep learning neural network 302 to produce a protocol classification label 402. More specifically, the protocol component 116 can feed the prescription document 202 to the input layer of the deep learning neural network 302. In various cases, the prescription document 202 can complete a forward pass through the one or more hidden layers of the deep learning neural network 302. In various aspects, the output layer of the deep learning neural network 302 can compute or calculate the protocol classification label 402 based on activation maps or feature maps produced by the one or more hidden layers of the deep learning neural network 302.

In various aspects, the protocol classification label 402 can be any suitable electronic data (e.g., can be one or more scalars, one or more vectors, one or more matrices, one or more tensors, one or more character strings, or any suitable combination thereof) that can represent, convey, or otherwise indicate a specific imaging protocol that is (in the opinion of the deep learning neural network 302) recited or otherwise called for in the prescription document 202. In particular, there can be a plurality of defined imaging protocols 404. In various instances, the plurality of defined imaging protocols 404 can comprise n protocols, for any suitable positive integer n>1: a defined imaging protocol 404(1) to a defined imaging protocol 404(n). In various cases, each of the plurality of defined imaging protocols can be or otherwise represent a distinct or unique configuration of scanning parameters that the medical imaging scanner 104 can possibly use to capture a medical image of the medical patient 106 (e.g., the defined imaging protocol 404(1) can be a first protocol that the medical imaging scanner 104 could possibly use to capture a medical image of the medical patient 106; the defined imaging protocol 404(n) can be an n-th protocol that the medical imaging scanner 104 could possibly use to capture a medical image of the medical patient 106).

In various aspects, the protocol classification label 402 can comprise a plurality of probability scores 406. In various instances, the plurality of probability scores 406 can respectively correspond (e.g., in one-to-one fashion) to the plurality of defined imaging protocols 404. Thus, since the plurality of defined imaging protocols 404 can comprise n protocols, the plurality of probability scores 406 can likewise comprise n scores; a probability score 406(1) to a probability score 406(n). In various cases, each of the plurality of probability scores 406 can be a real-valued scalar that indicates a likelihood (as inferred by the deep learning neural network 302) that the prescription document 202 recites, requests, or otherwise calls for a respective one of the plurality of defined imaging protocols 404. As a non-limiting example, the probability score 406(1) can correspond to the defined imaging protocol 404(1). Thus, the probability score 406(1) can be a first scalar estimated by the deep learning neural network 302 and whose value (e.g., ranging from 0 to 1, or from 0% to 100%) indicates a likelihood that the prescription document 202 requests that the defined imaging protocol 404(1) be performed on the medical patient 106. As another non-limiting example, the probability score 406(n) can correspond to the defined imaging protocol 404(n). So, the probability score 406(n) can be an n-th scalar estimated by the deep learning neural network 302 and whose value indicates a likelihood that the prescription document 202 requests that the defined imaging protocol 404(n) be performed on the medical patient 106.

Note that, in some cases, the plurality of probability scores 406 can be not independent of each other. As a non-limiting example, the plurality of probability scores 406 can be restricted such that their total sum can be unity (e.g., can be 1 or 100%). In such case, the deep learning neural network 302 can be considered as determining that the prescription document 202 requests or calls for only one of the plurality of defined imaging protocols 404 (e.g., whichever one of the plurality of defined imaging protocols 404 has the highest probability score can be considered as being indicated by the protocol classification label 402). However, in other cases, the plurality of probability scores 406 can be independent of each other. As a non-limiting example, each of the plurality of probability scores 406 can range from 0 (e.g., 0%) to 1 (e.g., 100%), regardless of the values of any others of the plurality of probability scores 406 (e.g., with no unity restriction on the total sum of the plurality of probability scores 406). In such case, the deep learning neural network 302 can be considered as being able to determine that the prescription document 202 requests or calls for the performance of multiple of the plurality of defined imaging protocols 404 on the medical patient 106 (e.g., whichever one or more of the plurality of defined imaging protocols 404 have probability scores that exceed any suitable threshold value can be considered as being indicated by the protocol classification label 402).

In any case, at least one of the plurality of defined imaging protocols 404 can be indicated by the protocol classification label 402 as being requested or called for by the prescription document 202, and such protocol can be referred to as the prescribed imaging protocol 304.

Now, consider FIG. 5. Rather than being configured as a text classifier, the deep learning neural network 302 can instead be configured as a large language model (LLM). In such case, the deep learning neural network 302 can comprise an encoder portion 502 and a synthesizer portion 504. In various cases, the encoder portion 502 can be considered as being upstream from the synthesizer portion 504. Equivalently, the synthesizer portion 504 can be considered as being downstream of the encoder portion 502.

In various aspects, the encoder portion 502 can exhibit any suitable deep learning internal architecture. Indeed, in various cases, the encoder portion 502 can have an input layer, one or more hidden layers, and an output layer. In various instances, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. As even another example, any of such input layer, one or more hidden layers, or output layer can be LSTM layers, whose learnable or trainable parameters can be input-state weight matrices or hidden-state weight matrices. As yet another example, any of such input layer, one or more hidden layers, or output layer can be transformer layers, whose learnable or trainable parameters can be single-head or multi-head attention blocks or other weight matrices. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

Likewise, in various instances, the synthesizer portion 504 can exhibit any suitable deep learning internal architecture. Indeed, in various cases, the synthesizer portion 504 can have an input layer, one or more hidden layers, and an output layer. In various instances, any of such layers can be coupled together by any suitable interneuron connections or interlayer connections (e.g., forward connections, skip connections, recurrent connections). Furthermore, in various cases, any of such layers can be any suitable types of neural network layers having any suitable learnable or trainable internal parameters (e.g., any of such input layer, one or more hidden layers, or output layer can be convolutional layers, dense layers, batch normalization layers, LSTM layers, or transformer layers). Further still, in various cases, any of such layers can be any suitable types of neural network layers having any suitable fixed or non-trainable internal parameters (e.g., any of such input layer, one or more hidden layers, or output layer can be non-linearity layers, padding layers, pooling layers, or concatenation layers).

Regardless of the specific internal architecture that is implemented within the encoder portion 502, the encoder portion 502 can be configured to receive textual data (which can be accompanied by any suitable numerical or graphical data) and to produce embeddings based on such inputted textual data. In contrast, regardless of the specific internal architecture that is implemented within the synthesizer portion 504, the synthesizer portion 504 can be configured to receive embeddings produced by the encoder portion 502 and to produce synthesized textual content based on such embeddings.

In various aspects, an embedding produced by the encoder portion 502 in response to a piece of inputted textual, numerical, or graphical data can be considered as any suitable mathematical quantity (e.g., scalar, vector, matrix, tensor, or any suitable combination thereof) that numerically represents at least some substantive or semantic aspect of that inputted textual, numerical, or graphical data in a low-dimensional fashion. In other words, the embedding can be smaller in terms of size or dimensionality (e.g., in some cases, one or more orders of magnitude smaller) than such inputted textual, numerical, or graphical data; but despite such smaller size, the embedding can nevertheless be considered as substantively or semantically representing such inputted textual, numerical, or graphical data. In still other words, the embedding can be considered as a latent vector representation of such inputted textual, numerical, or graphical data.

In any case, the deep learning neural network 302 can, in some instances, be structured as an LLM. As some non-limiting examples, the deep learning neural network 302 can be any of the following: ChatGPT; Gene.AI®; Ollama®; Bard®; Claude®; Seamless®; GitHub CoPilot®; or Amazon Code Whisperer®.

In various aspects, there can be a protocol identification prompt 506 that is feedable to the deep learning neural network 302. In various aspects, the protocol identification prompt 506 can be one or more unstructured or plain text sentences or sentence fragments that request or command identification of whichever imaging protocol that is conveyed by or recited in the prescription document 202. As a non-limiting example, the protocol identification prompt 506 can be the following sentence: “What imaging protocol, if any, of the medical imaging scanner 104 is indicated by the prescription document 202?” As another non-limiting example, the protocol identification prompt 506 can be the following sentence: “Identify what scanning protocol the prescription document 202 requests.”

Now, in various instances, the protocol component 116 can electronically execute the deep learning neural network 302 on the prescription document 202 and on the protocol identification prompt 506. In various cases, such execution can cause the deep learning neural network 302 to produce a protocol indication 508. More specifically, the protocol component 116 can concatenate the prescription document 202 and the protocol identification prompt 506 together. In various aspects, the protocol component 116 can feed that concatenation to the input layer of the encoder portion 502. In various aspects, that concatenation can complete a forward pass through the one or more hidden layers of the encoder portion 502. In various instances, the output layer of the encoder portion 502 can compute or otherwise calculate one or more embeddings (not shown), based on activation maps or feature maps provided by the one or more hidden layers of the encoder portion 502. In various cases, those one or more embeddings can be routed to the input layer of the synthesizer portion 504. In various aspects, those one or more embeddings can complete a forward pass through the one or more hidden layers of the synthesizer portion 504, and the output layer of the synthesizer portion 504 can compute or otherwise calculate the protocol indication 508 based on activation maps or feature maps provided by the one or more hidden layers of the synthesizer portion 504.

In various aspects, the protocol indication 508 can be one or more unstructured or plain text declarative sentences or sentence fragments that semantically answer or respond to the protocol identification prompt 506. That is, the protocol indication 508 can be synthesized text that names, states, or otherwise identifies a specific imaging protocol that (in the opinion of the deep learning neural network 302) is recited, called for, or otherwise prescribed in the prescription document 202, and such specific imaging protocol can be referred to as the prescribed imaging protocol 304.

Although FIGS. 4-5 pertain to embodiments in which the protocol component 116 utilizes a text classifier or an LLM to identify the prescribed imaging protocol 304, these are mere non-limiting examples for ease of explanation and illustration. In various other embodiments, the protocol component 116 can instead utilize any other suitable natural language processing techniques in order to identify the prescribed imaging protocol 304 from or based on the prescription document 202.

In any case, the protocol component 116 can identify the prescribed imaging protocol 304 based on the prescription document 202. Note that, in some cases, such identification can be due to the recitation of specific body parts, tissue types, or pathologies in the prescription document 202. Indeed, different imaging protocols can be considered as being specialized or tailored to capture images of different types of body parts. As a non-limiting example, a first imaging protocol might be optimized to capture images of heads or brains, whereas a second imaging protocol might be optimized to capture images of knee joints. Additionally, note that different imaging protocols can be considered as being specialized or tailored to capture images of different types of tissues, symptoms, pathologies, or other anatomical substances within a given body part. As a non-limiting example, a third imaging protocol might be optimized to capture images of healthy brains, whereas a fourth imaging protocol might be optimized to capture images of brains with lesions or tumors, and whereas a fifth imaging protocol might be optimized to capture images of brains with ischemic strokes. Accordingly, in some aspects, the prescription document 202 can recite or specify various body parts or pathology symptoms, the deep learning neural network 302 (or any other suitable natural language processing technique) can recognize which available protocol (e.g., which of 404) is mapped or correlated to those recited or specified body parts or pathology symptoms, and such protocol can be referred to or considered as the prescribed imaging protocol 304.

As a non-limiting example, the prescription document 202 can be or recite “rt. knee bone”, and the protocol component 116 can (e.g., via the deep learning neural network 302) interpret this to call for whatever imaging protocol whose settings are configured to investigate the bones associated with the right knee. As another non-limiting example, the prescription document 202 can be or recite “knee L cart”, and the protocol component 116 can interpret this to call for whatever imaging protocol whose settings are configured to investigate the cartilage associated with the left knee. As another non-limiting example, the prescription document 202 can be or recite “head i-stroke”, and the protocol component 116 can interpret this to call for whatever imaging protocol whose settings are configured to investigate ischemic strokes associated with the head.

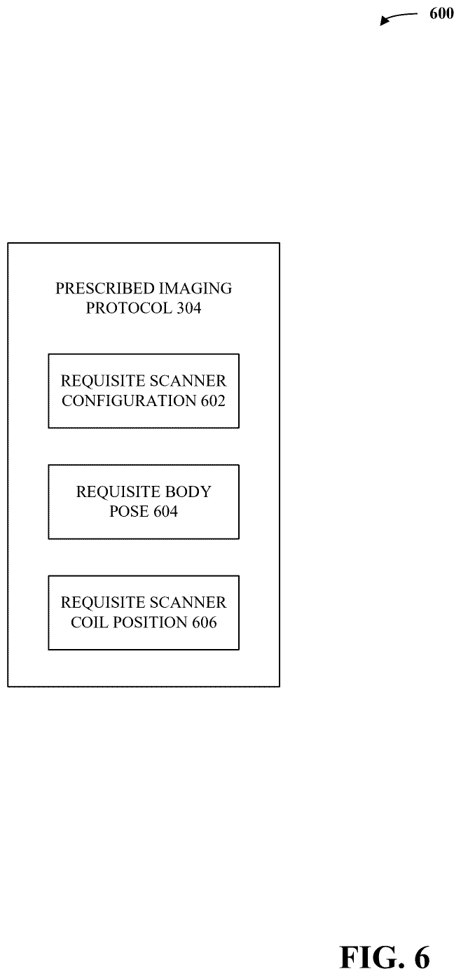

FIG. 6 illustrates an example, non-limiting block diagram 600 of the prescribed imaging protocol 304 in accordance with one or more embodiments described herein.

In various aspects, as shown, the prescribed imaging protocol 304 can comprise, specify, or otherwise be associated with a requisite scanner configuration 602. In various instances, the requisite scanner configuration 602 can be any suitable electronic data exhibiting any suitable format, size, or dimensionality (e.g., can be one or more scalars, one or more vectors, one or more matrices, one or more tensors, one or more character strings, or any suitable combination thereof) that is, indicates, or otherwise conveys the specific values or states that should be assigned to the scanning parameters of the medical imaging scanner 104 so as to cause the medical imaging scanner 104 to perform the prescribed imaging protocol 304. As a non-limiting example, the requisite scanner configuration 602 can indicate or specify what specific values or states should be assigned to the sequence-type parameter, to the slice thickness parameter, to the slice orientation parameter, to the FOV parameter, to the matrix size parameter, to the TR parameter, to the TE parameter, or to the NEX parameter of the medical imaging scanner 104 so that the medical imaging scanner 104 can perform the prescribed imaging protocol 304.

In various cases, as shown, the prescribed imaging protocol 304 can comprise, specify, or otherwise be associated with a requisite body pose 604. In various aspects, the requisite body pose 604 can be any suitable electronic data exhibiting any suitable format, size, or dimensionality (e.g., can be one or more scalars, one or more vectors, one or more matrices, one or more tensors, one or more character strings, or any suitable combination thereof) that indicates or otherwise conveys a specific physical orientation that the prescribed imaging protocol 304 assumes or is otherwise intended to be applied to. As a non-limiting example, the prescribed imaging protocol 304 can assume or otherwise be intended to be performed on patients whose bodies are oriented in a prone (e.g., lying face-down or belly-down) pose. As another non-limiting example, the prescribed imaging protocol 304 can assume or otherwise be intended to be performed on patients whose bodies are oriented in a supine (e.g., lying face-up or belly-up) pose. As even another non-limiting example, the prescribed imaging protocol 304 can assume or otherwise be intended to be performed on patients whose bodies are oriented in a right recumbent (e.g., lying on right side) pose. As still another non-limiting example, the prescribed imaging protocol 304 can assume or otherwise be intended to be performed on patients whose bodies are oriented in a left recumbent (e.g., lying on left side) pose. As yet another non-limiting example, the prescribed imaging protocol 304 can assume or otherwise be intended to be performed on patients whose bodies are oriented in a head-first (e.g., head entering scanner before feet) pose. As another non-limiting example, the prescribed imaging protocol 304 can assume or otherwise be intended to be performed on patients whose bodies are oriented in a feet-first (e.g., head entering scanner after feet) pose. In any case, if the prescribed imaging protocol 304 were to be performed on a patient whose body is not oriented in the requisite body pose 604, the prescribed imaging protocol 304 would not be able to be properly performed on that patient (e.g., any resulting scanned images would be incorrect or filled with artifacts).

In various aspects, as shown, the prescribed imaging protocol 304 can comprise, specify, or otherwise be associated with a requisite scanner coil position 606. Indeed, as mentioned above, the medical imaging scanner 104 can utilize or otherwise be associated with the scanner coil 122. In various aspects, the requisite scanner coil position 606 can be any suitable electronic data exhibiting any suitable format, size, or dimensionality (e.g., can be one or more scalars, one or more vectors, one or more matrices, one or more tensors, one or more character strings, or any suitable combination thereof) that indicates or otherwise conveys a specific on-body location of the scanner coil 122 that the prescribed imaging protocol 304 assumes or is otherwise intended to be applied to. As a non-limiting example, the prescribed imaging protocol 304 can assume or otherwise be intended to be performed on patients that are wearing the scanner coil 122 on their head. As another non-limiting example, the prescribed imaging protocol 304 can assume or otherwise be intended to be performed on patients that are wearing the scanner coil 122 on their right upper arm. As still another non-limiting example, the prescribed imaging protocol 304 can assume or otherwise be intended to be performed on patients that are wearing the scanner coil 122 on their left calf. In other words, the scanner coil position 606 can be considered as indicating a specific anatomy, and a laterality of that specific anatomy, that is supposed to be scanned by the medical imaging scanner 104. In any case, if the prescribed imaging protocol 304 were to be performed on a patient that is wearing the scanner coil 122 at some on-body location that is different from the requisite scanner coil position 606, the prescribed imaging protocol 304 would not be able to be properly performed on that patient (e.g., any resulting scanned images would be incorrect or filled with artifacts).

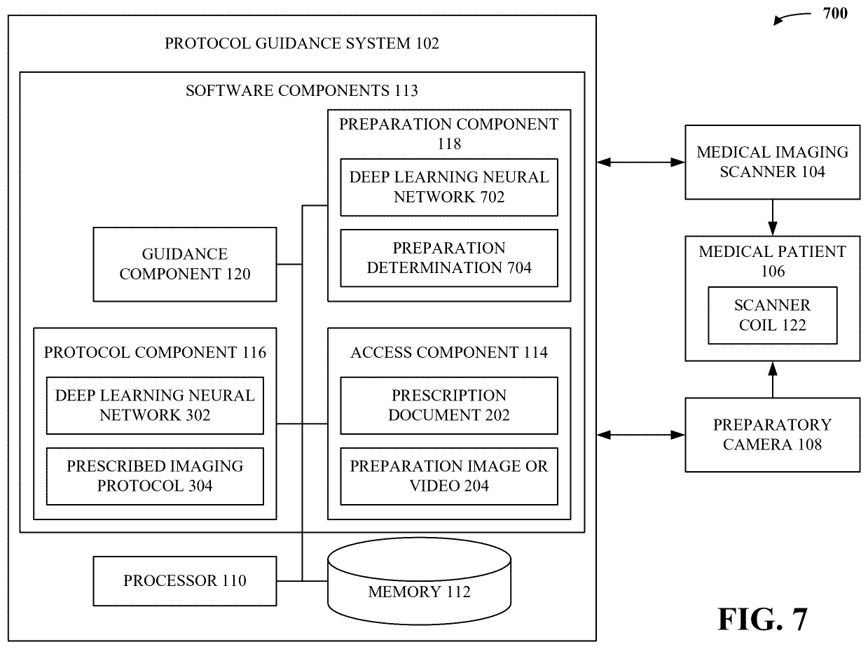

FIG. 7 illustrates a block diagram of an example, non-limiting system 700 including a second deep learning neural network and a preparation determination that can facilitate camera-based deep learning prediction and guidance for medical imaging protocols in accordance with one or more embodiments described herein. As shown, the system 700 can, in some cases, comprise the same components as the system 300, and can further comprise a deep learning neural network 702 and a preparation determination 704.

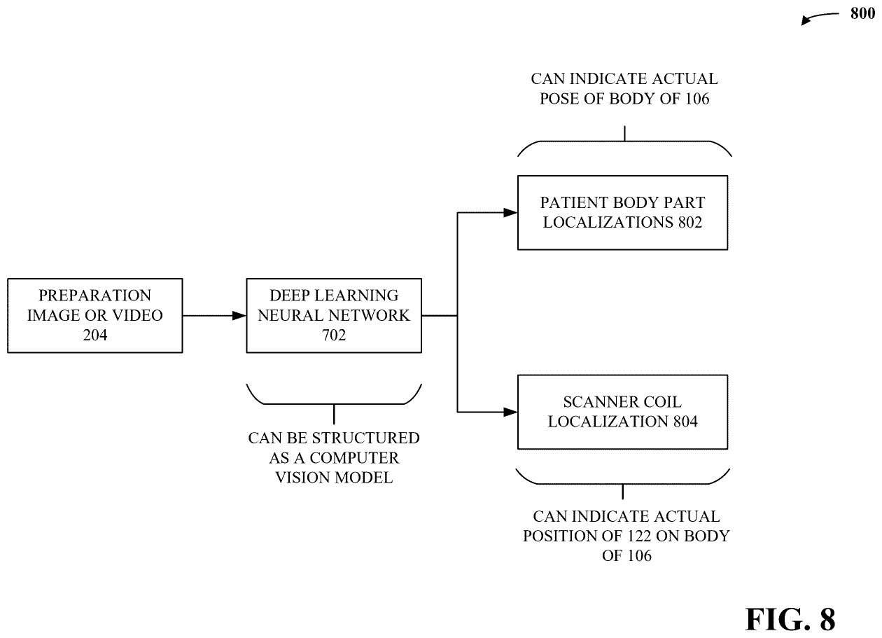

In various embodiments, the preparation component 118 can electronically store, electronically maintain, electronically control, or otherwise electronically access the deep learning neural network 702. In various cases, the preparation component 118 can leverage the deep learning neural network 702 so as to generate the preparation determination 704, based on the prescribed imaging protocol 304 and based on the preparation image or video 204. Non-limiting aspects are described with respect to FIGS. 8-12.

FIGS. 8-12 illustrate example, non-limiting block diagrams showing how the preparation determination 704 can be obtained in accordance with one or more embodiments described herein.

First, consider the non-limiting block diagram 800 of FIG. 8. In various embodiments, as shown, the deep learning neural network 702 can be configured as a computer vision model having any suitable deep learning internal architecture. For instance, in various cases, the deep learning neural network 702 can have an input layer, one or more hidden layers, and an output layer. In various aspects, any of such layers can be coupled together by any suitable interneuron connections or interlayer connections, such as forward connections, skip connections, or recurrent connections. Furthermore, in various cases, any of such layers can be any suitable types of neural network layers having any suitable learnable or trainable internal parameters. For example, any of such input layer, one or more hidden layers, or output layer can be convolutional layers, whose learnable or trainable parameters can be convolutional kernels. As another example, any of such input layer, one or more hidden layers, or output layer can be dense layers, whose learnable or trainable parameters can be weight matrices or bias values. As still another example, any of such input layer, one or more hidden layers, or output layer can be batch normalization layers, whose learnable or trainable parameters can be shift factors or scale factors. As even another example, any of such input layer, one or more hidden layers, or output layer can be LSTM layers, whose learnable or trainable parameters can be input-state weight matrices or hidden-state weight matrices. As yet another example, any of such input layer, one or more hidden layers, or output layer can be transformer layers, whose learnable or trainable parameters can be single-head or multi-head attention blocks or other weight matrices. Further still, in various cases, any of such layers can be any suitable types of neural network layers having any suitable fixed or non-trainable internal parameters. For example, any of such input layer, one or more hidden layers, or output layer can be non-linearity layers, padding layers, pooling layers, or concatenation layers.

Regardless of the specific internal architecture (e.g., of the specific numbers, types, or organizations of layers) that is implemented in the deep learning neural network 702, the deep learning neural network 702 can be configured to receive as input visual data and to produce as output various localizations based on that inputted visual data. Accordingly, the preparation component 118 can electronically execute the deep learning neural network 702 on the preliminary image or video 204, and such execution can cause the deep learning neural network 702 to produce a set of patient body part localizations 802 or a scanner coil localization 804. More specifically, the preparation component 118 can feed the preparation image or video 204 to the input layer of the deep learning neural network 702. In various cases, the preparation image or video 204 can complete a forward pass through the one or more hidden layers of the deep learning neural network 702. In various aspects, the output layer of the deep learning neural network 702 can compute or calculate the set of patient body part localizations 802 or the scanner coil localization 804, based on activation maps or feature maps produced by the one or more hidden layers of the deep learning neural network 702.

In various embodiments, the set of patient body part localizations 802 can comprise any suitable number of patient body part localizations. In various aspects, each of the set of patient body part localizations 802 can be any suitable electronic data exhibiting any suitable format, size, or dimensionality (e.g., can be one or more scalars, one or more vectors, one or more matrices, one or more tensors, or any suitable combination thereof) that indicates or otherwise represents a location (as inferred by the deep learning neural network 702) of a respective body part of the medical patient 106 within the preparation image or video 204. As a non-limiting example, a first patient body part localization of the set of patient body part localizations 802 can correspond to a right eye of the medical patient 106. Accordingly, the first patient body part localization can be: landmark coordinates that indicate where (e.g., in terms of two-dimensional Cartesian coordinates) the right eye of the medical patient 106 is located within the preparation image or video 204; a bounding box that circumscribes the right eye of the medical patient 106 within the preparation image or video 204; or a segmentation mask that encompasses or covers the right eye of the medical patient 106 within the preparation image or video 204. As another non-limiting example, a second patient body part localization of the set of patient body part localizations 802 can correspond to a left ankle of the medical patient 106. Accordingly, the second patient body part localization can be: landmark coordinates that indicate where (e.g., in terms of two-dimensional Cartesian coordinates) the left ankle of the medical patient 106 is located within the preparation image or video 204; a bounding box that circumscribes the left ankle of the medical patient 106 within the preparation image or video 204; or a segmentation mask that encompasses or covers the left ankle of the medical patient 106 within the preparation image or video 204. As even another non-limiting example, a third patient body part localization of the set of patient body part localizations 802 can correspond to a nose of the medical patient 106. Accordingly, the third patient body part localization can be: landmark coordinates that indicate where (e.g., in terms of two-dimensional Cartesian coordinates) the nose of the medical patient 106 is located within the preparation image or video 204; a bounding box that circumscribes the nose of the medical patient 106 within the preparation image or video 204; or a segmentation mask that encompasses or covers the nose of the medical patient 106 within the preparation image or video 204. In any case, the set of patient body part localizations 802 can be considered as indicating or showing where (in the opinion of the deep learning neural network 702) various body parts of the medical patient 106 are actually located or positioned as the medical patient 106 prepares or waits to be scanned by the medical imaging scanner 104.

In various embodiments, the scanner coil localization 804 can be any suitable electronic data exhibiting any suitable format, size, or dimensionality (e.g., can be one or more scalars, one or more vectors, one or more matrices, one or more tensors, or any suitable combination thereof) that indicates or otherwise represents a location (as inferred by the deep learning neural network 702) of the scanner coil 122 within the preparation image or video 204. As a non-limiting example, the scanner coil localization 804 can be landmark coordinates that indicate where (e.g., in terms of two-dimensional Cartesian coordinates) the scanner coil 122 is located within the preparation image or video 204. As another non-limiting example, the scanner coil localization 804 can be a bounding box that circumscribes the scanner coil 122 within the preparation image or video 204. As yet another non-limiting example, the scanner coil localization 804 can be a segmentation mask that encompasses or covers the scanner coil 122 within the preparation image or video 204. In any case, the scanner coil localization 804 can be considered as indicating or showing where (in the opinion of the deep learning neural network 702) the medical patient 106 is actually wearing the scanner coil 122 as their prepare or wait to be scanned by the medical imaging scanner 104.

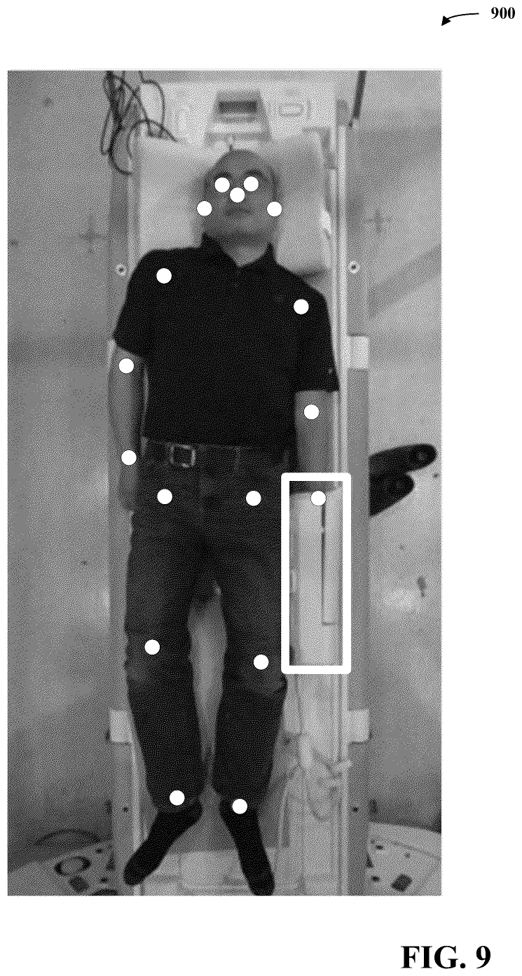

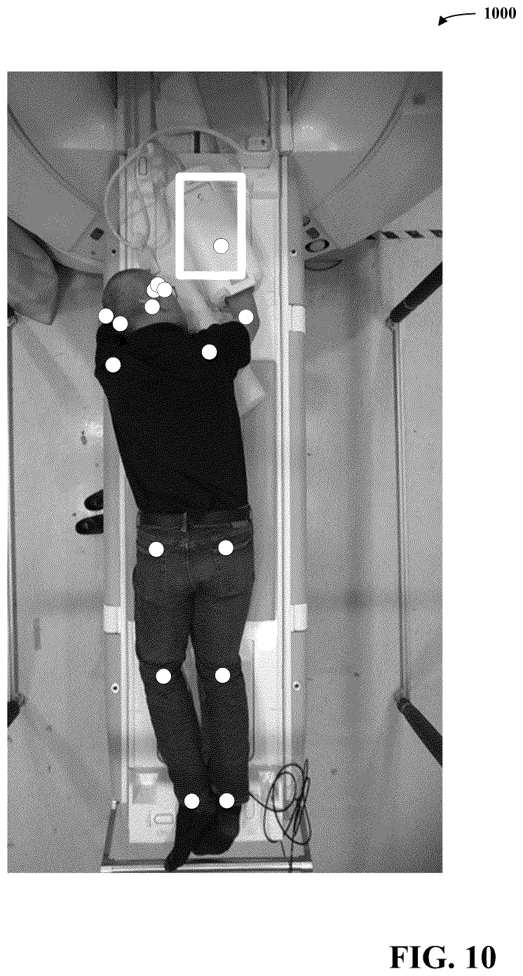

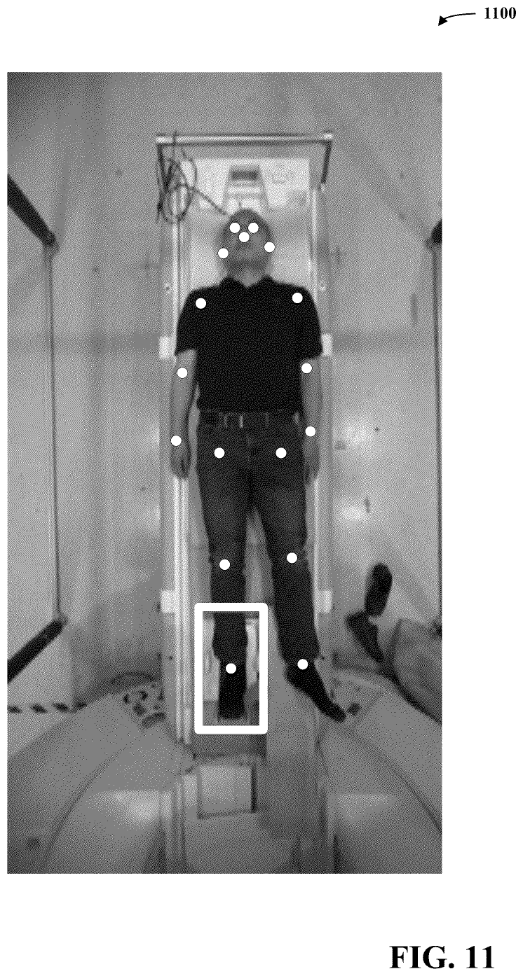

FIGS. 9-11 depict example, non-limiting embodiments of the preparation image or video 204 overlaid with example, non-limiting embodiments of the set of patient body part localizations 802 and the scanner coil localization 804. Specifically, FIG. 9 shows an image 900 that depicts a male patient lying in a supine pose on a table of an MRI scanner. The white circles in FIG. 9 indicate the set of patient body part localizations 802. That is, in the non-limiting example of FIG. 9, the set of patient body part localizations 802 are landmark coordinates that demarcate or denote intra-image locations of respective body parts of the male patient. In the non-limiting example of FIG. 9, those body parts include: right eye; left eye; nose; right car; left car; right shoulder; left shoulder; right elbow; left elbow; right wrist; left wrist; right hip; left hip; right knee; left knee; right ankle; and left ankle. Furthermore, the white rectangle in FIG. 9 indicates the scanner coil localization 804. That is, in the non-limiting example of FIG. 9, the scanner coil localization 804 is a bounding box that circumscribes a scanner coil worn by the male patient. As shown, the scanner coil of FIG. 9 is worn on the left hand of the male patient. FIG. 10 shows an image 1000 that depicts the same male patient, who is now lying in a prone pose and wearing the scanner coil on his right hand. FIG. 11 shows an image 1100 that depicts the same male patient, who is lying in a supine pose and wearing a partially-enclosing scanner coil on his right ankle.

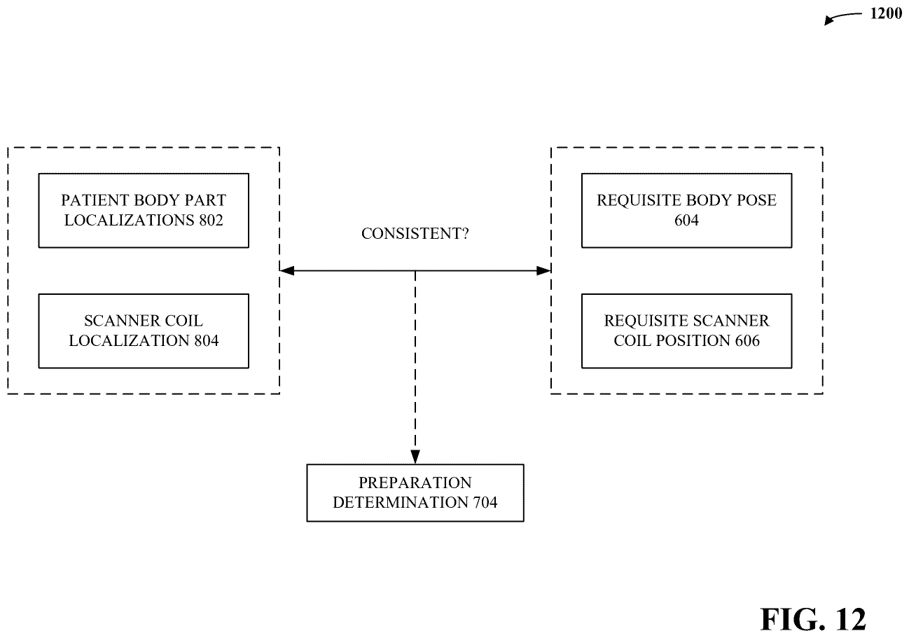

Now, consider the non-limiting block diagram 1200 of FIG. 12. In various embodiments, the preparation determination 704 can be any suitable electronic data having any suitable format, size, or dimensionality (e.g., can be one or more scalars, one or more vectors, one or more matrices, one or more tensors, one or more character strings, or any suitable combination thereof) that binarily or dichotomously indicates whether or not the medical patient 106 is properly prepared for performance of the prescribed imaging protocol 304. In various aspects, the preparation component 118 can electronically generate the preparation determination 704, by comparing: the set of patient body part localizations 802 and the scanner coil localization 804; to the requisite body pose 604 and the requisite scanner coil position 606.

As mentioned above, the requisite body pose 604 can be considered as indicating whatever specific physical orientation or pose that the body of the medical patient 106 should exhibit, so that the prescribed imaging protocol 304 can be properly performed on the medical patient 106. In various instances, the set of patient body part localizations 802 can be considered as collectively indicating or conveying whatever physical orientation or pose that the body of the medical patient 106 actually exhibits at a current, present, or otherwise recent time (e.g., the preparatory camera 108 can capture real-time images or videos of the medical patient 106).

As a non-limiting example, suppose that the set of patient body part localizations 802 indicate that the eyes, noses, and cars of the medical patient 106 are located above (from the perspective of the preparatory camera 108) the ankles of the medical patient 106. In such case, the preparation component 118 can conclude from the set of patient body part localizations 802 that the medical patient 106 is currently or presently oriented in a head-first pose.

As another non-limiting example, suppose that the set of patient body part localizations 802 indicate that the eyes, noses, and cars of the medical patient 106 are located below (from the perspective of the preparatory camera 108) the ankles of the medical patient 106. In such case, the preparation component 118 can conclude from the set of patient body part localizations 802 that the medical patient 106 is currently or presently oriented in a feet-first pose.

As yet another non-limiting example, suppose that the set of patient body part localizations 802 indicate that the right shoulder, right hip, right elbow, and right knee of the medical patient 106 are located rightward (from the perspective of the preparatory camera 108) of the left shoulder, left hip, left elbow, and left knee of the medical patient 106. In such case, the preparation component 118 can conclude from the set of patient body part localizations 802 that the medical patient 106 is currently or presently oriented in a prone pose.

As even another non-limiting example, suppose that the set of patient body part localizations 802 indicate that the right shoulder, right hip, right elbow, and right knee of the medical patient 106 are located leftward (from the perspective of the preparatory camera 108) of the left shoulder, left hip, left elbow, and left knee of the medical patient 106. In such case, the preparation component 118 can conclude from the set of patient body part localizations 802 that the medical patient 106 is currently or presently oriented in a supine pose.

As still another non-limiting example, suppose that the set of patient body part localizations 802 indicate that the right and left shoulders of the medical patient 106 are located (from the perspective of the preparatory camera 108) on top of each other and that the eyes or nose of the medical patient 106 are located rightward (from the perspective of the preparatory camera 108) of the shoulders or hips. In such case, the preparation component 118 can conclude from the set of patient body part localizations 802 that the medical patient 106 is currently or presently oriented in a left recumbent pose.

As another non-limiting example, suppose that the set of patient body part localizations 802 indicate that the right and left shoulders of the medical patient 106 are located (from the perspective of the preparatory camera 108) on top of each other and that the eyes or nose of the medical patient 106 are located leftward (from the perspective of the preparatory camera 108) of the shoulders or hips. In such case, the preparation component 118 can conclude from the set of patient body part localizations 802 that the medical patient 106 is currently or presently oriented in a right recumbent pose.

Furthermore, as mentioned above, the requisite scanner coil position 606 can be considered as indicating whatever specific on-body location at which the medical patient 106 should wear the scanner coil 122, so that the prescribed imaging protocol 304 can be properly performed on the medical patient 106. In various instances, the scanner coil localization 804 can, in conjunction with the set of patient body part localizations 802, be considered as indicating or conveying whatever on-body location at which the medical patient 106 is currently or presently wearing the scanner coil 122.

As a non-limiting example, suppose that the scanner coil localization 804 coincides with (e.g., overlaps with, is on top of, or is within any suitable threshold proximity of) whichever of the set of patient body part localizations 802 that corresponds to the left wrist of the medical patient 106 (such as is shown in FIG. 9). In such case, the preparation component 118 can conclude that the medical patient 106 is currently or presently wearing the scanner coil 122 on their left wrist or hand.

As another non-limiting example, suppose that the scanner coil localization 804 coincides with (e.g., overlaps with, is on top of, or is within any suitable threshold proximity of) whichever of the set of patient body part localizations 802 that corresponds to the right wrist of the medical patient 106 (such as is shown in FIG. 10). In such case, the preparation component 118 can conclude that the medical patient 106 is currently or presently wearing the scanner coil 122 on their right wrist or hand.

As still another non-limiting example, suppose that the scanner coil localization 804 coincides with (e.g., overlaps with, is on top of, or is within any suitable threshold proximity of) whichever of the set of patient body part localizations 802 that corresponds to the right ankle of the medical patient 106 (such as is shown in FIG. 11). In such case, the preparation component 118 can conclude that the medical patient 106 is currently or presently wearing the scanner coil 122 on their right ankle or foot.

As even another non-limiting example, suppose that the scanner coil localization 804 coincides with (e.g., overlaps with, is on top of, or is within any suitable threshold proximity of) whichever of the set of patient body part localizations 802 that correspond to the eyes, cars, or nose of the medical patient 106. In such case, the preparation component 118 can conclude that the medical patient 106 is currently or presently wearing the scanner coil 122 on their head.

Accordingly, in various aspects, the preparation component 118 can generate the preparation determination 704, by comparing the requisite body pose 604 to the actual body pose of the medical patient 106 that is conveyed by the set of patient body part localizations 802, and by comparing the requisite scanner coil position 606 to the actual on-body scanner coil position collectively conveyed by the set of patient body part localizations 802 and the scanner coil localization 804. In various instances, the preparation determination 704 can indicate that the medical patient 106 is properly prepared for the prescribed imaging protocol 304, if both the requisite body pose 604 and the requisite scanner coil position 606 are satisfied (e.g., if the actual body pose of the medical patient 106 matches the requisite body pose 604, and if the actual on-body location of the scanner coil 122 matches the requisite scanner coil position 606). In contrast, the preparation determination 704 can indicate that the medical patient 106 is not yet properly prepared for the prescribed imaging protocol 304, if either of the requisite body pose 604 and the requisite scanner coil position 606 is unsatisfied (e.g., if the actual body pose of the medical patient 106 does not match the requisite body pose 604, or if the actual on-body location of the scanner coil 122 does not match the requisite scanner coil position 606).

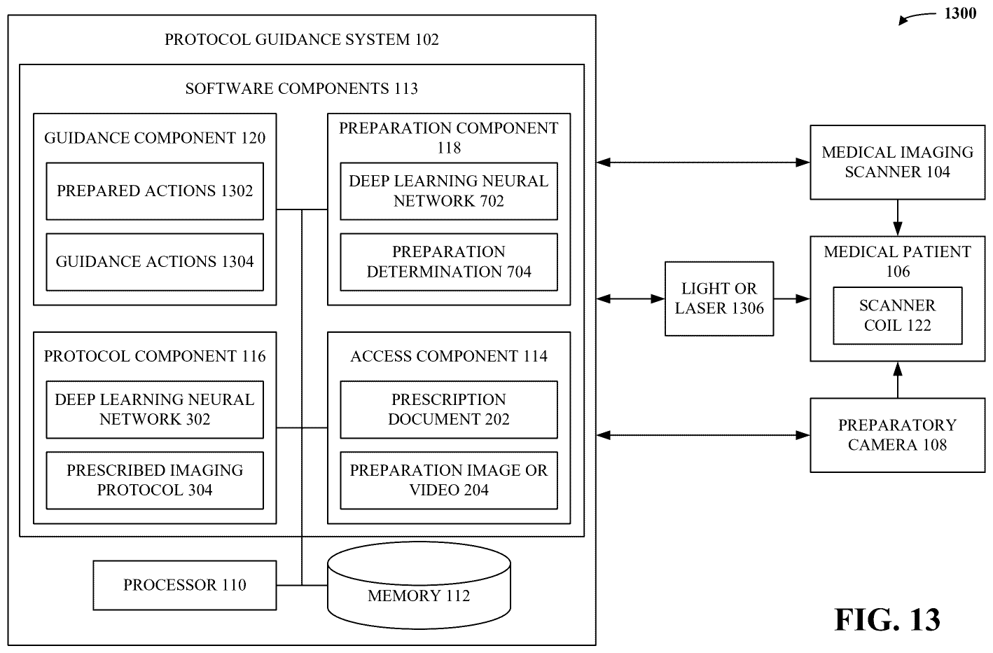

FIG. 13 illustrates a block diagram of an example, non-limiting system 1300 including one or more prepared actions and one or more guidance actions that can facilitate camera-based deep learning prediction and guidance for medical imaging protocols in accordance with one or more embodiments described herein. As shown, the system 1300 can, in some cases, comprise the same components as the system 700, and can further comprise one or more prepared actions 1302, one or more guidance actions 1304, and a light or laser 1306.

In various embodiments, the guidance component 120 can electronically initiate or otherwise perform any suitable actions, based on the preparation determination 704. In particular, if the preparation determination 704 indicates that the medical patient 106 is prepared for the prescribed imaging protocol 304, then the guidance component 120 can perform or initiate the one or more prepared actions 1302. On the other hand, if the preparation determination 704 instead indicates that the medical patient 106 is not prepared for the prescribed imaging protocol 304, then the guidance component 120 can perform or initiate the one or more guidance actions 1304.

In various aspects, the one or more prepared actions 1302 can include electronically transmitting to the medical imaging scanner 104 an instruction or command to begin performance of the prescribed imaging protocol 304. This can be considered as an auto-start functionality of the medical imaging scanner 104 that is triggered by the preparation determination 704 indicating that the medical patient 106 is prepared for the prescribed imaging protocol 304.

In various other aspects, the one or more prepared actions 1302 can instead include electronically transmitting to the medical imaging scanner 104 (or to any other computerized workstation associated with the medical imaging scanner 104) a first notification that can be displayed or rendered on the GUI of the medical imaging scanner 104 (or on a GUI of the other computerized workstation), such that the first notification can be viewed by the user or operator of the medical imaging scanner 104. In various instances, the first notification can indicate that the medical patient 106 is prepared for commencement of the prescribed imaging protocol 304, and the first notification can further request or prompt the user or operator for permission to begin performing the prescribed imaging protocol 304. In some cases, the user or operator can (via the GUI) press, click, or otherwise select any suitable invocable GUI button or element to signify their permission, and the medical imaging scanner 104 can, in response to such pressing, clicking, or selecting, begin to perform the prescribed imaging protocol 304 on the medical patient 106.

In various aspects, the one or more guidance actions 1304 can include electronically transmitting to the medical imaging scanner 104 (or to any other computerized workstation associated with the medical imaging scanner 104) a second notification that can be displayed or rendered on the GUI of the medical imaging scanner 104 (or on a GUI of the other computerized workstation), such that the second notification can be viewed by the user or operator of the medical imaging scanner 104. In various instances, the second notification can indicate that the medical patient 106 is not yet prepared for commencement of the prescribed imaging protocol 304. In various cases, the second notification can indicate or otherwise convey a specific reason explaining why the medical patient 106 is not yet prepared or ready. As a non-limiting example, if the preparation component 118 concludes that the requisite body pose 604 is not satisfied, the second notification can indicate or otherwise convey that the medical patient 106 will not be prepared or ready until their body is oriented in compliance with the requisite body pose 604. As another non-limiting example, if the preparation component 118 concludes that the requisite scanner coil position 606 is not satisfied, the second notification can indicate or otherwise convey that the medical patient 106 will not be prepared or ready until the scanner coil 122 is worn on their body in compliance with the requisite scanner coil position 606. In some aspects, the second notification can comprise any suitable illustrations or diagrams that visually indicate the requisite body pose 604 or the requisite scanner coil position 606, so as to aid the understanding of the user or operator (e.g., the second notification can include a rendition of the preparation image or video 204 on which the requisite scanner coil position 606 can be highlighted or otherwise visually demarcated).

In some instances, the one or more guidance actions 1304 can include controllably adjusting the light or laser 1306. Indeed, in various cases, the light or laser 1306 can be any suitable physically actuatable, orientable, or aimable light beam emitting device or laser beam emitting device that is associated with the medical imaging scanner 104. In some aspects, the light or laser 1306 can be physically built or integrated into the medical imaging scanner 104. In other aspects, the light or laser 1306 can be physically remote or separate from, but nevertheless in the same room as, the medical imaging scanner 104. In yet other aspects, the light or laser 1306 can be physically remote or separate from, and in a different room than, the medical imaging scanner 104. In some instances, the light or laser 1306 can be physically coupled or integrated into the preparatory camera 108, such that the light or laser 1306 can be able to shine onto whatever is within a field of view of the preparatory camera 108. In any case, the light or laser 1306 can be able to selectively emit a visible light beam or visible laser beam of any suitable color or intensity onto any suitable external body part of the medical patient 106 as they are preparing or waiting to be scanned by the medical imaging scanner 104 (e.g., waiting in or on the gantry, table, or bay of the medical imaging scanner 104; or waiting in an adjacent donning/doffing room associated with the medical imaging scanner 104). If the preparation component 118 concludes that the requisite scanner coil position 606 is not satisfied, the one or more guidance actions 1304 can include instructing, commanding, or otherwise causing the light or laser 1306 to aim (e.g., the light or laser 1306 can be movable by any suitable actuators, such as servo motors) at and conspicuously illuminate whichever body part of the medical patient 106 corresponds to the requisite scanner coil position 606. As a non-limiting example, suppose that the requisite scanner coil position 606 is the right hand of the medical patient 106. In such case, the set of patient body part localizations 802 can be considered as indicating where the right hand of the medical patient 106 is currently or presently located in the real-world with respect to the medical imaging scanner 104, with respect to the preparatory camera 108, or with respect to the light or laser 1306, and the guidance component 120 can cause the light or laser 1306 to shine a visible light beam or laser beam onto the actual, real-world right hand of the medical patient 106. As another non-limiting example, suppose that the requisite scanner coil position 606 is the left knee of the medical patient 106. In such case, the set of patient body part localizations 802 can be considered as indicating where the left knee of the medical patient 106 is currently or presently located in the real-world with respect to the medical imaging scanner 104, with respect to the preparatory camera 108, or with respect to the light or laser 1306, and the guidance component 120 can cause the light or laser 1306 to shine a visible light beam or laser beam onto the actual, real-world left knee of the medical patient 106. In any case, the light beam or laser beam emitted by the light or laser 1306 can be considered as conspicuously or noticeably showing the user or operator (or even the medical patient 106 themself) where the scanner coil 122 should be moved to on the body of the medical patient 106.

In various aspects, the one or more guidance actions 1304 can be considered as showing or teaching the user or operator of the medical imaging scanner 104 how to rectify the body pose or scanner coil position of the medical patient 106 so as to make them ready for the prescribed imaging protocol 304. After the user or operator makes an adjustment to the medical patient 106, the preparatory camera 108 can capture or record a new preparation image or video (e.g., a new or later instantiation of 204), the preparation component 118 can generate a new preparation determination (e.g., a new or later instantiation of 704), and the guidance component 120 can repeat the one or more prepared actions 1302 or the one or more guidance actions 1304 accordingly.

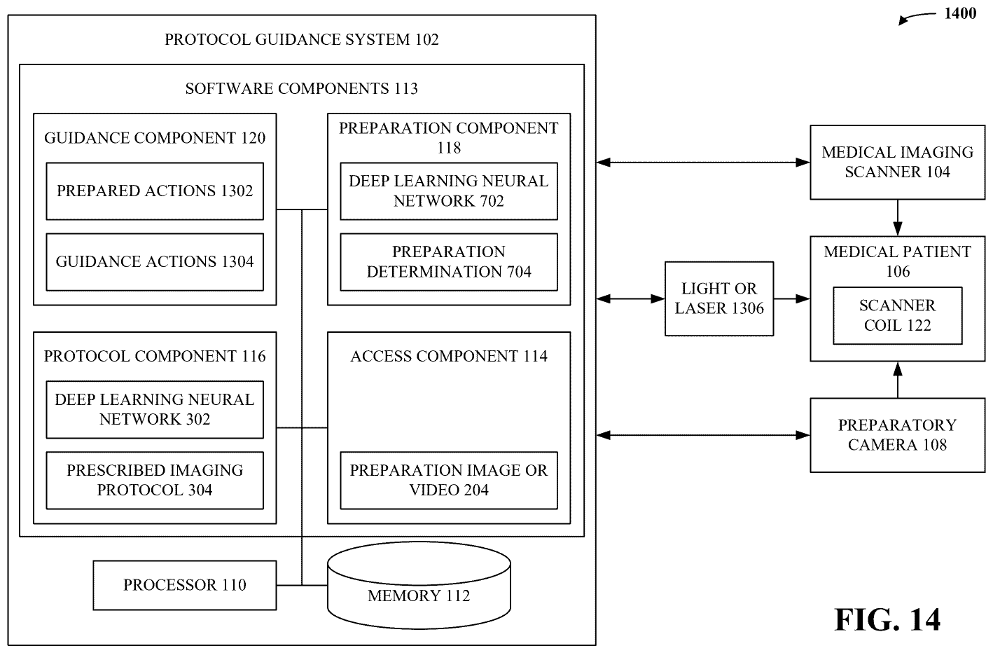

FIG. 14 illustrates a block diagram of an example, non-limiting system 1400 excluding a prescription document that can facilitate camera-based deep learning prediction and guidance for medical imaging protocols in accordance with one or more embodiments described herein. As shown, the system 1400 can, in some cases, comprise the same components as the system 1300, but can exclude or omit the prescription document 202.

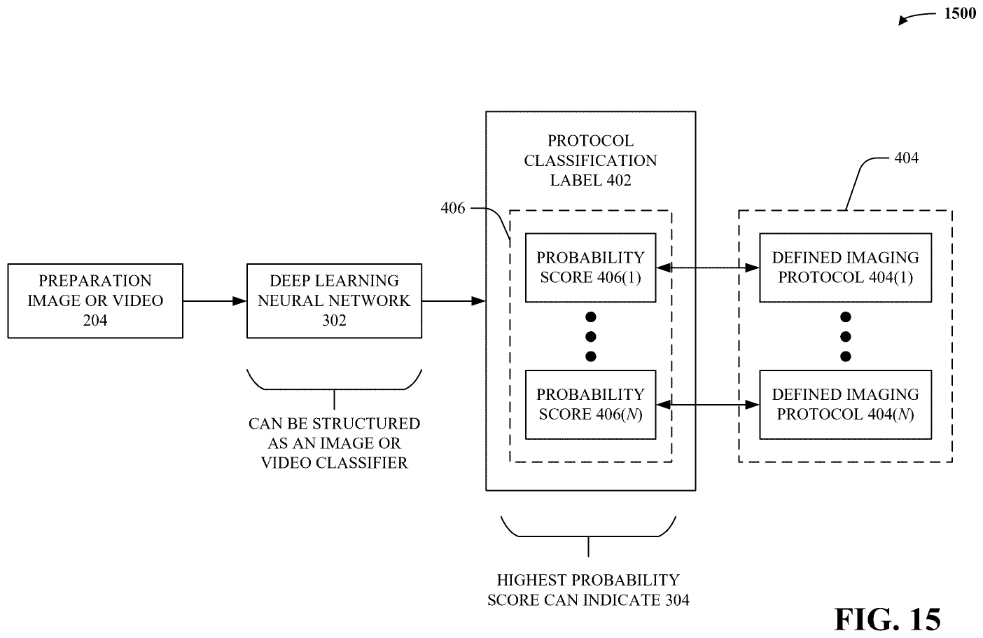

In various embodiments, the prescription document 202 might be unavailable. In such situations, the protocol component 116 can nevertheless leverage the deep learning neural network 302 to identify the prescribed imaging protocol 304. Non-limiting aspects are described with respect to FIG. 15.

FIG. 15 illustrates an example, non-limiting block diagram 1500 showing how the prescribed imaging protocol 304 can be determined in the absence of a prescription document in accordance with one or more embodiments described herein.

In various embodiments, as shown, the deep learning neural network 302 can be configured as an image classifier, rather than as a text classifier or LLM. Even in such case, the deep learning neural network 302 can have any suitable deep learning internal architecture (e.g., any suitable types of layers arranged in any suitable format or layout and having any suitable trainable or non-trainable internal parameters).

Regardless of its specific internal architecture, the deep learning neural network 302 can, in the non-limiting example of FIG. 15, be configured to receive as input visual data and to produce as output a classification label for that inputted visual data. Accordingly, the protocol component 116 can electronically execute the deep learning neural network 302 on the preparation image or video 204 (rather than on the prescription document 202), and such execution can cause the deep learning neural network 302 to produce the protocol classification label 402. That is, the protocol component 116 can feed the preparation image or video 204 to the input layer of the deep learning neural network 302, the preparation image or video 204 can complete a forward pass through the one or more hidden layers of the deep learning neural network 302, and the output layer of the deep learning neural network 302 can compute or calculate the protocol classification label 402 based on activation maps or feature maps produced by the one or more hidden layers of the deep learning neural network 302. In various aspects, the protocol classification label 402 can be as described above (e.g., can comprise the plurality of probability scores 406 which can respectively correspond to the plurality of defined imaging protocols 404).

In situations where the deep learning neural network 302 generates the protocol classification label 402 based on the preparation image or video 204, the deep learning neural network 302 can be considered as inferring the prescribed imaging protocol 304 based on whatever initial or coarse on-body position at which the medical patient 106 is wearing the scanner coil 122. As a non-limiting example, if the user or operator originally or initially places the scanner coil 122 within any suitable threshold proximity of the right hand of the medical patient 106, it can be reasonably expected (and the deep learning neural network 302 can thus infer or predict) that the prescribed imaging protocol 304 is whichever protocol corresponds or is tailored to right hands. If multiple of such protocols exist, any suitable disambiguation technique can be used (e.g., random selection among those multiple protocols). As another non-limiting example, if the user or operator places the scanner coil 122 within any suitable threshold proximity of the left knee of the medical patient 106, it can be reasonably expected (and the deep learning neural network 302 can thus infer or predict) that the prescribed imaging protocol 304 is whichever protocol corresponds or is tailored to left knees. Again, if multiple of such protocols exist, any suitable disambiguation technique can be used. In any case, situations where the prescription document 202 is absent can be overcome or handled by inferring the prescribed imaging protocol 304 from the preparation image or video 204 (e.g., derived from where the scanner coil 122 is initially or originally placed onto the medical patient 106).

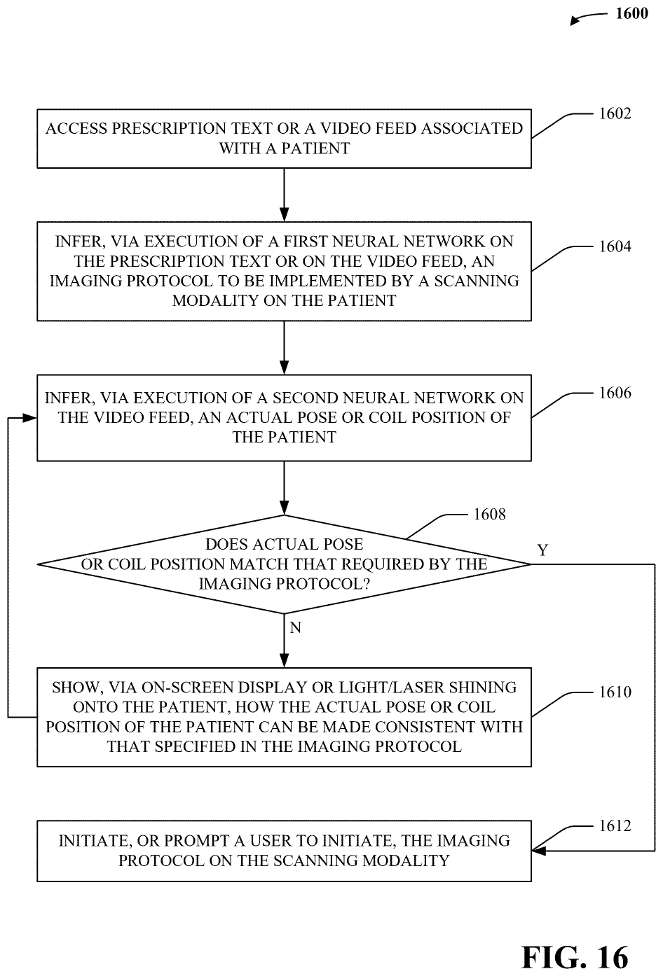

FIG. 16 illustrates a flow diagram of an example, non-limiting computer-implemented method 1600 that can facilitate camera-based deep learning prediction and guidance for medical imaging protocols in accordance with one or more embodiments described herein. In various cases, the protocol guidance system 102 can facilitate the computer-implemented method 1600.

In various embodiments, act 1602 can include accessing, by a device (e.g., via 114) operatively coupled to a processor (e.g., 110), prescription text (e.g., 202) or a video feed (e.g., 204) associated with a patient (e.g., 106).

In various aspects, act 1604 can include inferring, by the device (e.g., via 116) and via execution of a first neural network (e.g., 302) on the prescription text or on the video feed, an imaging protocol (e.g., 304) to be implemented by a scanning modality (e.g., 104) on the patient.

In various instances, act 1606 can include inferring, by the device (e.g., via 118) and via execution of a second neural network (e.g., 702) on the video feed, an actual pose (e.g., collectively indicated by 802) or coil position (e.g., collectively indicated by 802 and 804) of the patient.

In various cases, act 1608 can include determining, by the device (e.g., via 118), whether the actual pose or coil position match that (e.g., 604 or 606) required by the imaging protocol. If not, the computer-implemented method 1600 can proceed to act 1610. If so, the computer-implemented method 1600 can instead proceed to act6 1612.

In various aspects, act 1610 can include showing, by the device (e.g., via 120) and via an on-screen display or a light/laser shining onto the patient, how the actual pose or coil position of the patient can be made consistent or compliant with that specified in the imaging protocol. In various cases, the computer-implemented method 1600 can proceed back to act 1606 (e.g., the video feedback can be continually updated as time progresses).

In various instances, act 1612 can include initiating, by the device (e.g., via 120) the imaging protocol on the scanning modality, or prompting, by the device (e.g., via 120), a user to initiate the imaging protocol on the scanning modality.

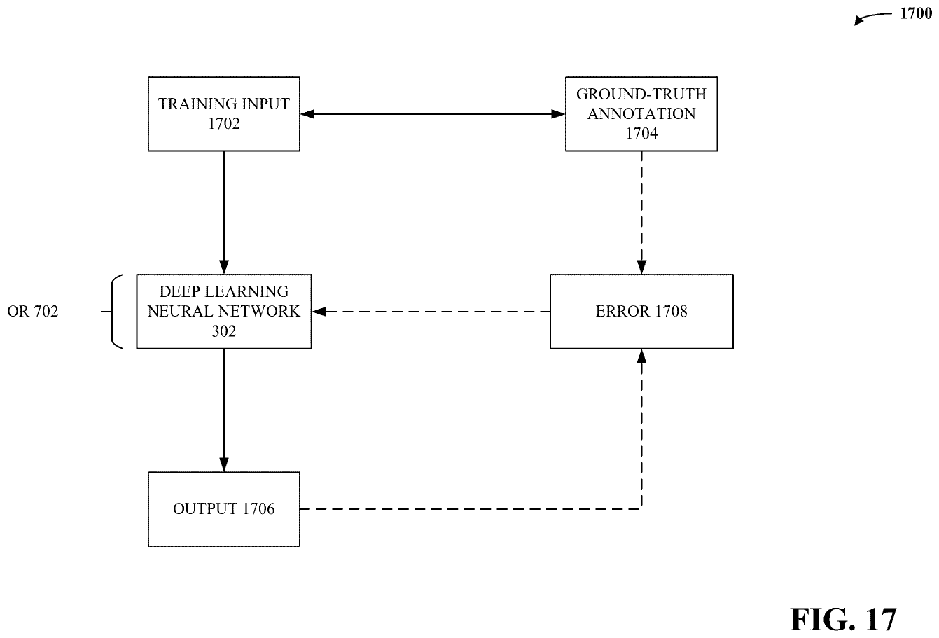

In order for the herein-described prediction and guidance of medical imaging protocols to be accurate, correct, or reliable, the various machine learning models described herein can first undergo training. A non-limiting example of such training is described with respect to FIG. 17.

FIG. 17 illustrates an example, non-limiting block diagram 1700 showing how various artificial intelligence models can be trained in accordance with one or more embodiments described herein.

In various aspects, prior to beginning training, the trainable internal parameters (e.g., convolutional kernels, weight matrices, bias values) of whatever artificial intelligence model is being trained (e.g., the deep learning neural network 302, the deep learning neural network 702) can be initialized in any suitable fashion (e.g., via random initialization).

In various embodiments, there can be a training input 1702 and a ground-truth annotation 1704. When it is desired to train the deep learning neural network 302, the training input 1702 can be any suitable training prescription document (or a concatenation thereof with any suitable training protocol identification prompt as appropriate; or any suitable training image or video as appropriate), and the ground-truth annotation 1704 can be whatever correct or accurate protocol classification label (or correct or accurate protocol indication as appropriate) is known or deemed to correspond to the training input 1702. When it is desired to train the deep learning neural network 702, the training input 1702 can be any suitable training image or video, and the ground-truth annotation 1704 can be whatever correct or accurate patient body part localizations or correct or accurate scanner coil localization that are known or deemed to correspond to the training input 1702.

In any case, the artificial intelligence model that is being trained can be executed on the training input 1702, thereby causing that artificial intelligence model to produce an output 1706. More specifically, in some cases, the training input 1702 can be fed or routed to the input layer of the artificial intelligence model, the training input 1702 can complete a forward pass through the one or more hidden layers of the artificial intelligence model, and the output layer of the artificial intelligence model can compute the output 1706 based on activation maps or feature maps provided by the one or more hidden layers of the artificial intelligence model.

Note that the format, size, or dimensionality of the output 1706 can be dictated by the number, arrangement, sizes, or other characteristics of the neurons, convolutional kernels, LSTM weights, or other internal parameters of the output layer (or of any other layers) of the artificial intelligence model. Accordingly, the output 1706 can be forced to have any desired format, size, or dimensionality, by adding, removing, or otherwise adjusting characteristics of the output layer (or of any other layers) of the artificial intelligence model.

In various aspects, if the output 1706 is produced by the deep learning neural network 302, the output 1706 can be considered as the predicted or inferred protocol classification label (or the predicted or inferred protocol indication as appropriate) that the deep learning neural network 302 believes should correspond to the training input 1702. If the output 1706 is produced by the deep learning neural network 702, the output 1706 can be considered as the predicted or inferred patient body part localizations or the predicted or inferred scanner coil localization that the deep learning neural network 702 believes should correspond to the training input 1702. Note that, if the artificial intelligence model that is being trained has so far undergone no or little training, then the output 1706 can be highly inaccurate. In other words, the output 1706 can be very different from the ground-truth annotation 1704.

In various aspects, an error 1708 (e.g., mean absolute error (MAE), mean squared error (MSE), cross-entropy error) between the output 1706 and the ground-truth annotation 1704 can be computed. In various instances, the trainable internal parameters of the artificial intelligence model can be incrementally updated via backpropagation (e.g., stochastic gradient descent) based on the error 1708.

In various cases, such execution-and-update procedure can be repeated for any suitable number of input-annotation pairs. This can ultimately cause the trainable internal parameters of the artificial intelligence model (e.g., of the deep learning neural network 302, of the deep learning neural network 702) to become iteratively optimized for accurately performing its inferencing task (e.g., protocol classification or determination; body part or scanner coil localization). In various aspects, any suitable training batch sizes, any suitable error/loss functions, or any suitable training termination criteria can be utilized during such training.

Although the herein disclosure mainly describes the various artificial intelligence models as being trained in supervised fashion, this is a mere non-limiting example for case of explanation and illustration. In various embodiments, any other suitable training paradigms can be used to train the deep learning neural network 302 or the deep learning neural network 702, such as unsupervised training or reinforcement learning, any of which may be federated or non-federated.

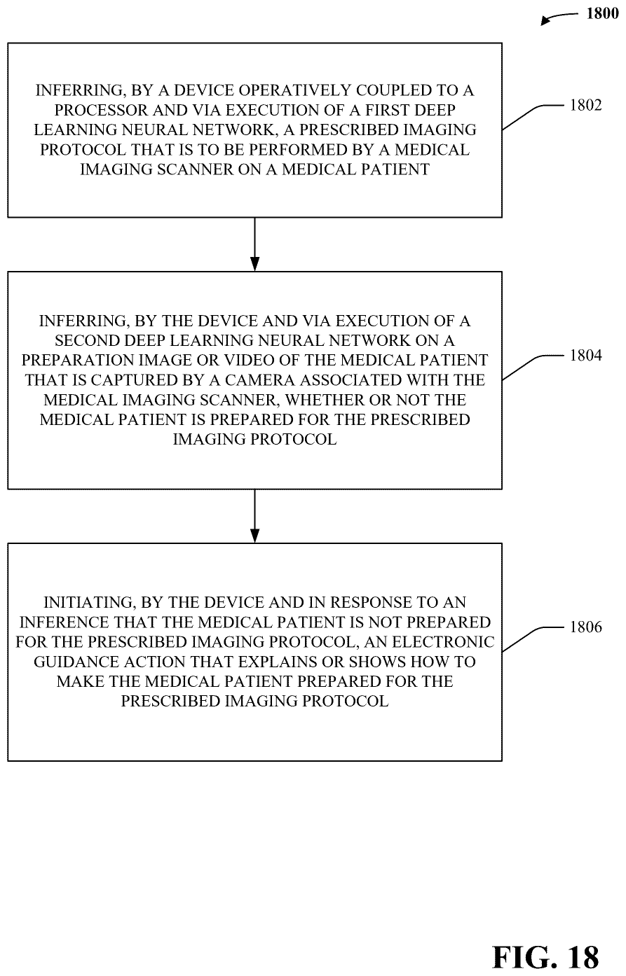

FIG. 18 illustrates a flow diagram of an example, non-limiting computer-implemented method 1800 that can facilitate camera-based deep learning prediction and guidance for medical imaging protocols in accordance with one or more embodiments described herein. In various cases, the protocol guidance system 102 can facilitate the computer-implemented method 1800.

In various embodiments, act 1802 can include inferring, by a device (e.g., via 116) operatively coupled to a processor (e.g., 110) and via execution of a first deep learning neural network (e.g., 302), a prescribed imaging protocol (e.g., 304) that is to be performed by a medical imaging scanner (e.g., 104) on a medical patient (e.g., 106).

In various aspects, act 1804 can include inferring, by the device (e.g., via 118) and via execution of a second deep learning neural network (e.g., 702) on a preliminary image or video (e.g., 204) of the medical patient that is captured by a camera (e.g., 108) associated with the medical imaging scanner, whether or not the medical patient is prepared for the prescribed imaging protocol.

In various instances, act 1806 can include initiating, by the device (e.g., via 120) and in response to an inference (e.g., 704) that the medical patient is not prepared for the prescribed imaging protocol, an electronic guidance action (e.g., 1304) that explains or shows how to make the medical patient prepared for the prescribed imaging protocol.

Although not explicitly shown in FIG. 18, the computer-implemented method 1800 can include: rendering, by the device (e.g., via 120), in response to an inference (e.g., 704) that the medical patient is prepared for the prescribed imaging protocol, and on a graphical user-interface of the medical imaging scanner, a notification (e.g., included in 1302) indicating that the prescribed imaging protocol is ready to be performed and requesting a user of the medical imaging scanner to approve performance of the prescribed imaging protocol; or instructing, by the device (e.g., via 120), the medical imaging scanner to perform the prescribed imaging protocol.

Although not explicitly shown in FIG. 18, the first deep learning neural network can be a large language model that: receives as input: a textual prescription (e.g., 202) written by a medical professional attending to the medical patient; and a protocol identification prompt (e.g., 506); and produces as output synthesized text (e.g., 508) indicating the prescribed imaging protocol.

Although not explicitly shown in FIG. 18, the first deep learning neural network can be an image classifier that: receives as input the preliminary image or video of the medical patient captured by the camera; and produces as output a classification label (e.g., 402) indicating the prescribed imaging protocol.

Although not explicitly shown in FIG. 18, the second deep learning neural network can receive as input the preliminary image and produce as output a localization (e.g., 802) indicating a current body pose or orientation of the medical patient, and the device can infer that the medical patient is not prepared, in response to the current body pose or orientation not matching a requisite body pose or orientation (e.g., 604) specified in the prescribed imaging protocol.

Although not explicitly shown in FIG. 18, the second deep learning neural network can receive as input the preliminary image and produce as output a localization (e.g., 804) indicating a current scanner coil position on the medical patient, and the device can infer that the medical patient is not prepared, in response to the current scanner coil position not matching a requisite scanner coil position (e.g., 606) specified in the prescribed imaging protocol.

Although not explicitly shown in FIG. 18, the electronic guidance action can comprise rendering, on a graphical user-interface of the medical imaging scanner: a current body pose or orientation (e.g., 802) or a current scanner coil position (e.g., collectively by 802 and 804) of the medical patient, inferred by the second deep learning neural network; and a requisite body pose or orientation (e.g., 604) or a requisite scanner coil position (e.g., 606), specified in the prescribed imaging protocol.

Although not explicitly shown in FIG. 18, a current scanner coil location (e.g., collectively by 802 and 804) of the medical patient inferred by the second deep learning neural network can fail to match a requisite scanner coil position (e.g., 606) specified in the prescribed imaging protocol, and the electronic guidance action can include shining a light or laser (e.g., 1306) associated with the medical imaging scanner onto the body of the medical patient in accordance with the requisite scanner coil position.

Although not explicitly shown in FIG. 18, the camera can be located in a separate room than the medical imaging scanner.

Various embodiments described herein can involve a computer program product for facilitating camera-based deep learning prediction and guidance for medical imaging protocols. In various aspects, the computer program product can comprise a non-transitory computer-readable memory (e.g., 112) having program instructions embodied therewith. In various instances, the program instructions can be executable by a processor (e.g., 110) to cause the processor to infer, via execution of a first deep learning neural network (e.g., 302) on a physician prescription (e.g., 202) corresponding to a medical patient (e.g., 106) or on a video feed (e.g., 204) depicting the medical patient, a prescribed imaging protocol (e.g., 304) that is to be performed by a magnetic resonance imaging (MRI) scanner (e.g., 104) on the medical patient. In various cases, the program instructions can be further executable to cause the processor to infer, via execution of a second deep learning neural network (e.g., 702) on the video feed, whether or not an MRI coil position (e.g., indicated by 802 and 804 collectively) on the medical patient fails to match a requisite MRI coil position (e.g., 606) specified in the prescribed imaging protocol (e.g., such inference represented by 704). In various aspects, the program instructions can be further executable to cause the processor to cause, in response to an inference that the MRI coil position does not match the requisite MRI coil position, an actuatable light or laser (e.g., 1306) associated with the MRI scanner to shine onto the body of the medical patient, thereby visibly lighting the requisite MRI coil position on the medical patient (e.g., such action represented by 1304). In various instances, the program instructions can be further executable to cause the processor to cause, in response to an inference that the MRI coil position does match the requisite MRI coil position, the MRI scanner to perform the prescribed imaging protocol (e.g., such action represented by 1302).

Although various embodiments described herein mainly describe embodiments in which scanner coils are localized by the deep learning neural network 702 and compared to requisite scanner coil positions specified by or otherwise corresponding to the prescribed imaging protocol 304, these are mere non-limiting examples for ease of explanation and illustration. In various cases, the deep learning neural network 702 can be trained or configured to localize any other suitable objects of interest that are wearable by a medical patient and that pertain to or otherwise affect medical imaging scans (e.g., can be trained or configured to localize radiation bibs or shields worn by medical patients; can be trained or configured to localize dental mouth openers or lip retractors worn by medical patients).

In various instances, machine learning algorithms or models can be implemented in any suitable way to facilitate any suitable aspects described herein. To facilitate some of the above-described machine learning aspects of various embodiments, consider the following discussion of artificial intelligence (AI). Various embodiments described herein can employ artificial intelligence to facilitate automating one or more features or functionalities. The components can employ various AI-based schemes for carrying out various embodiments/examples disclosed herein. In order to provide for or aid in the numerous determinations (e.g., determine, ascertain, infer, calculate, predict, prognose, estimate, derive, forecast, detect, compute) described herein, components described herein can examine the entirety or a subset of the data to which it is granted access and can provide for reasoning about or determine states of the system or environment from a set of observations as captured via events or data. Determinations can be employed to identify a specific context or action, or can generate a probability distribution over states, for example. The determinations can be probabilistic; that is, the computation of a probability distribution over states of interest based on a consideration of data and events. Determinations can also refer to techniques employed for composing higher-level events from a set of events or data.

Such determinations can result in the construction of new events or actions from a set of observed events or stored event data, whether or not the events are correlated in close temporal proximity, and whether the events and data come from one or several event and data sources. Components disclosed herein can employ various classification (explicitly trained (e.g., via training data) as well as implicitly trained (e.g., via observing behavior, preferences, historical information, receiving extrinsic information, and so on)) schemes or systems (e.g., support vector machines, neural networks, expert systems, Bayesian belief networks, fuzzy logic, data fusion engines, and so on) in connection with performing automatic or determined action in connection with the claimed subject matter. Thus, classification schemes or systems can be used to automatically learn and perform a number of functions, actions, or determinations.

A classifier can map an input attribute vector, z=(z₁, z₂, z₃, z₄, zₙ), to a confidence that the input belongs to a class, as by f(z)=confidence (class). Such classification can employ a probabilistic or statistical-based analysis (e.g., factoring into the analysis utilities and costs) to determinate an action to be automatically performed. A support vector machine (SVM) can be an example of a classifier that can be employed. The SVM operates by finding a hyper-surface in the space of possible inputs, where the hyper-surface attempts to split the triggering criteria from the non-triggering events. Intuitively, this makes the classification correct for testing data that is near, but not identical to training data. Other directed and undirected model classification approaches include, e.g., naïve Bayes, Bayesian networks, decision trees, neural networks, fuzzy logic models, or probabilistic classification models providing different patterns of independence, any of which can be employed. Classification as used herein also is inclusive of statistical regression that is utilized to develop models of priority.

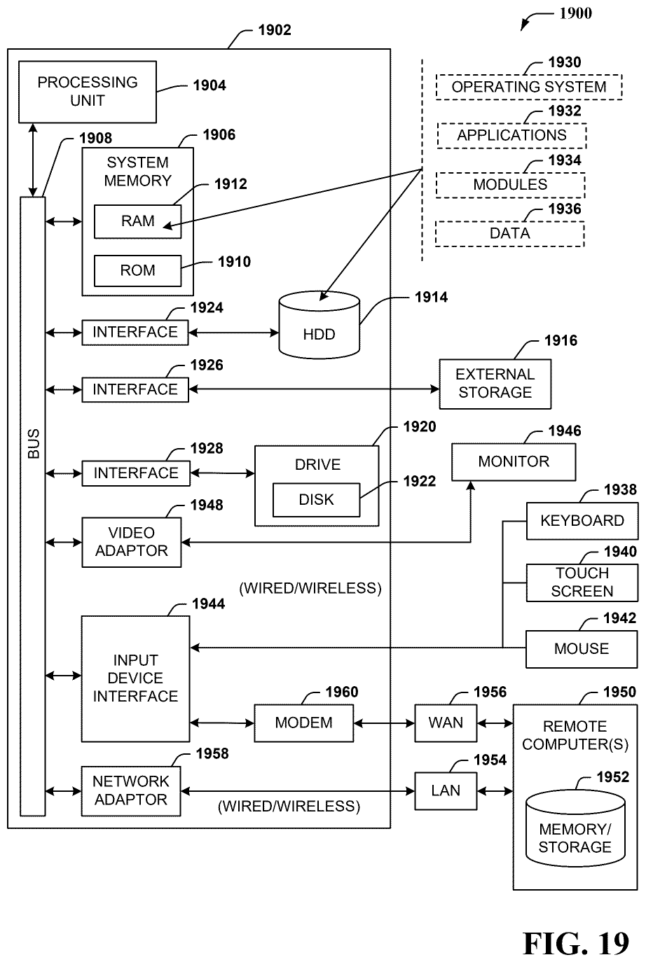

In order to provide additional context for various embodiments described herein, FIG. 19 and the following discussion are intended to provide a brief, general description of a suitable computing environment 1900 in which the various embodiments of the embodiment described herein can be implemented. While the embodiments have been described above in the general context of computer-executable instructions that can run on one or more computers, those skilled in the art will recognize that the embodiments can be also implemented in combination with other program modules or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multi-processor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable storage media, machine-readable storage media, or communications media, which two terms are used herein differently from one another as follows. Computer-readable storage media or machine-readable storage media can be any available storage media that can be accessed by the computer and includes both volatile and nonvolatile media, removable and non-removable media. By way of example, and not limitation, computer-readable storage media or machine-readable storage media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 19, the example environment 1900 for implementing various embodiments of the aspects described herein includes a computer 1902, the computer 1902 including a processing unit 1904, a system memory 1906 and a system bus 1908. The system bus 1908 couples system components including, but not limited to, the system memory 1906 to the processing unit 1904. The processing unit 1904 can be any of various commercially available processors. Dual microprocessors and other multi-processor architectures can also be employed as the processing unit 1904.

The system bus 1908 can be any of several types of bus structure that can further interconnect to a memory bus (with or without a memory controller), a peripheral bus, and a local bus using any of a variety of commercially available bus architectures. The system memory 1906 includes ROM 1910 and RAM 1912. A basic input/output system (BIOS) can be stored in a non-volatile memory such as ROM, erasable programmable read only memory (EPROM), EEPROM, which BIOS contains the basic routines that help to transfer information between elements within the computer 1902, such as during startup. The RAM 1912 can also include a high-speed RAM such as static RAM for caching data.

The computer 1902 further includes an internal hard disk drive (HDD) 1914 (e.g., EIDE, SATA), one or more external storage devices 1916 (e.g., a magnetic floppy disk drive (FDD) 1916, a memory stick or flash drive reader, a memory card reader, etc.) and a drive 1920, e.g., such as a solid state drive, an optical disk drive, which can read or write from a disk 1922, such as a CD-ROM disc, a DVD, a BD, etc. Alternatively, where a solid state drive is involved, disk 1922 would not be included, unless separate. While the internal HDD 1914 is illustrated as located within the computer 1902, the internal HDD 1914 can also be configured for external use in a suitable chassis (not shown). Additionally, while not shown in environment 1900, a solid state drive (SSD) could be used in addition to, or in place of, an HDD 1914. The HDD 1914, external storage device(s) 1916 and drive 1920 can be connected to the system bus 1908 by an HDD interface 1924, an external storage interface 1926 and a drive interface 1928, respectively. The interface 1924 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1902, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1912, including an operating system 1930, one or more application programs 1932, other program modules 1934 and program data 1936. All or portions of the operating system, applications, modules, or data can also be cached in the RAM 1912. The systems and methods described herein can be implemented utilizing various commercially available operating systems or combinations of operating systems.

Computer 1902 can optionally comprise emulation technologies. For example, a hypervisor (not shown) or other intermediary can emulate a hardware environment for operating system 1930, and the emulated hardware can optionally be different from the hardware illustrated in FIG. 19. In such an embodiment, operating system 1930 can comprise one virtual machine (VM) of multiple VMs hosted at computer 1902. Furthermore, operating system 1930 can provide runtime environments, such as the Java runtime environment or the .NET framework, for applications 1932. Runtime environments are consistent execution environments that allow applications 1932 to run on any operating system that includes the runtime environment. Similarly, operating system 1930 can support containers, and applications 1932 can be in the form of containers, which are lightweight, standalone, executable packages of software that include, e.g., code, runtime, system tools, system libraries and settings for an application.

Further, computer 1902 can be enable with a security module, such as a trusted processing module (TPM). For instance with a TPM, boot components hash next in time boot components, and wait for a match of results to secured values, before loading a next boot component. This process can take place at any layer in the code execution stack of computer 1902, e.g., applied at the application execution level or at the operating system (OS) kernel level, thereby enabling security at any level of code execution.

A user can enter commands and information into the computer 1902 through one or more wired/wireless input devices, e.g., a keyboard 1938, a touch screen 1940, and a pointing device, such as a mouse 1942. Other input devices (not shown) can include a microphone, an infrared (IR) remote control, a radio frequency (RF) remote control, or other remote control, a joystick, a virtual reality controller or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1904 through an input device interface 1944 that can be coupled to the system bus 1908, but can be connected by other interfaces, such as a parallel port, an IEEE 1394 serial port, a game port, a USB port, an IR interface, a BLUETOOTH® interface, etc.

A monitor 1946 or other type of display device can be also connected to the system bus 1908 via an interface, such as a video adapter 1948. In addition to the monitor 1946, a computer typically includes other peripheral output devices (not shown), such as speakers, printers, etc.

The computer 1902 can operate in a networked environment using logical connections via wired or wireless communications to one or more remote computers, such as a remote computer(s) 1950. The remote computer(s) 1950 can be a workstation, a server computer, a router, a personal computer, portable computer, microprocessor-based entertainment appliance, a peer device or other common network node, and typically includes many or all of the elements described relative to the computer 1902, although, for purposes of brevity, only a memory/storage device 1952 is illustrated. The logical connections depicted include wired/wireless connectivity to a local area network (LAN) 1954 or larger networks, e.g., a wide area network (WAN) 1956. Such LAN and WAN networking environments are commonplace in offices and companies, and facilitate enterprise-wide computer networks, such as intranets, all of which can connect to a global communications network, e.g., the Internet.

When used in a LAN networking environment, the computer 1902 can be connected to the local network 1954 through a wired or wireless communication network interface or adapter 1958. The adapter 1958 can facilitate wired or wireless communication to the LAN 1954, which can also include a wireless access point (AP) disposed thereon for communicating with the adapter 1958 in a wireless mode.

When used in a WAN networking environment, the computer 1902 can include a modem 1960 or can be connected to a communications server on the WAN 1956 via other means for establishing communications over the WAN 1956, such as by way of the Internet. The modem 1960, which can be internal or external and a wired or wireless device, can be connected to the system bus 1908 via the input device interface 1944. In a networked environment, program modules depicted relative to the computer 1902 or portions thereof, can be stored in the remote memory/storage device 1952. It will be appreciated that the network connections shown are example and other means of establishing a communications link between the computers can be used.

When used in either a LAN or WAN networking environment, the computer 1902 can access cloud storage systems or other network-based storage systems in addition to, or in place of, external storage devices 1916 as described above, such as but not limited to a network virtual machine providing one or more aspects of storage or processing of information. Generally, a connection between the computer 1902 and a cloud storage system can be established over a LAN 1954 or WAN 1956 e.g., by the adapter 1958 or modem 1960, respectively. Upon connecting the computer 1902 to an associated cloud storage system, the external storage interface 1926 can, with the aid of the adapter 1958 or modem 1960, manage storage provided by the cloud storage system as it would other types of external storage. For instance, the external storage interface 1926 can be configured to provide access to cloud storage sources as if those sources were physically connected to the computer 1902.

The computer 1902 can be operable to communicate with any wireless devices or entities operatively disposed in wireless communication, e.g., a printer, scanner, desktop or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

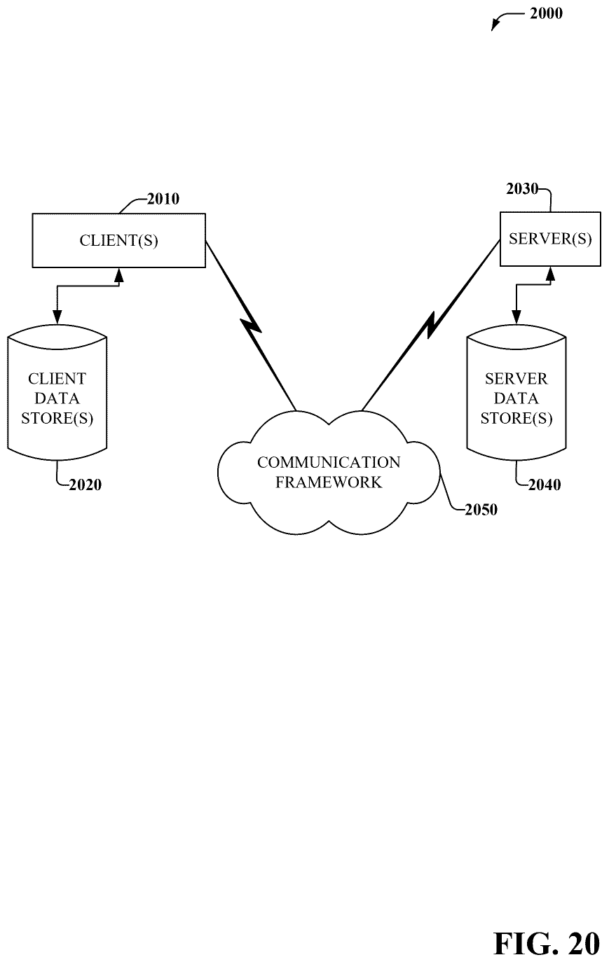

FIG. 20 is a schematic block diagram of a sample computing environment 2000 with which the disclosed subject matter can interact. The sample computing environment 2000 includes one or more client(s) 2010. The client(s) 2010 can be hardware or software (e.g., threads, processes, computing devices). The sample computing environment 2000 also includes one or more server(s) 2030. The server(s) 2030 can also be hardware or software (e.g., threads, processes, computing devices). The servers 2030 can house threads to perform transformations by employing one or more embodiments as described herein, for example. One possible communication between a client 2010 and a server 2030 can be in the form of a data packet adapted to be transmitted between two or more computer processes. The sample computing environment 2000 includes a communication framework 2050 that can be employed to facilitate communications between the client(s) 2010 and the server(s) 2030. The client(s) 2010 are operably connected to one or more client data store(s) 2020 that can be employed to store information local to the client(s) 2010. Similarly, the server(s) 2030 are operably connected to one or more server data store(s) 2040 that can be employed to store information local to the servers 2030.

Various embodiments may be a system, a method, an apparatus or a computer program product at any possible technical detail level of integration. The computer program product can include a computer readable storage medium (or media) having computer readable program instructions thereon for causing a processor to carry out aspects of various embodiments. The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium can also include the following: a portable computer diskette, a hard disk, a solid state drive such as M.2 (including non-volatile memory express (NVMe) or serial advanced technology attachment (SATA)), a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network or a wireless network. The network can comprise copper transmission cables, optical transmission fibers, wireless transmission, routers, firewalls, switches, gateway computers or edge servers. A network adapter card or network interface in each computing/processing device receives computer readable program instructions from the network and forwards the computer readable program instructions for storage in a computer readable storage medium within the respective computing/processing device. Computer readable program instructions for carrying out operations of various embodiments can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform various aspects.

Various aspects are described herein with reference to flowchart illustrations or block diagrams of methods, apparatus (systems), and computer program products according to various embodiments. It will be understood that each block of the flowchart illustrations or block diagrams, and combinations of blocks in the flowchart illustrations or block diagrams, can be implemented by computer readable program instructions. These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart or block diagram block or blocks. These computer readable program instructions can also be stored in a computer readable storage medium that can direct a computer, a programmable data processing apparatus, or other devices to function in a particular manner, such that the computer readable storage medium having instructions stored therein comprises an article of manufacture including instructions which implement aspects of the function/act specified in the flowchart or block diagram block or blocks. The computer readable program instructions can also be loaded onto a computer, other programmable data processing apparatus, or other device to cause a series of operational acts to be performed on the computer, other programmable apparatus or other device to produce a computer implemented process, such that the instructions which execute on the computer, other programmable apparatus, or other device implement the functions/acts specified in the flowchart or block diagram block or blocks.

The flowcharts and block diagrams in the Figures illustrate the architecture, functionality, and operation of possible implementations of systems, methods, and computer program products according to various embodiments. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams or flowchart illustration, and combinations of blocks in the block diagrams or flowchart illustration, can be implemented by special purpose hardware-based systems that perform the specified functions or acts or carry out combinations of special purpose hardware and computer instructions.

While the subject matter has been described above in the general context of computer-executable instructions of a computer program product that runs on a computer or computers, those skilled in the art will recognize that this disclosure also can or can be implemented in combination with other program modules. Generally, program modules include routines, programs, components, data structures, etc. that perform particular tasks or implement particular abstract data types. Moreover, those skilled in the art will appreciate that various aspects can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments in which tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, or a computer. By way of illustration, both an application running on a server and the server can be a component. One or more components can reside within a process or thread of execution and a component can be localized on one computer or distributed between two or more computers. In another example, respective components can execute from various computer readable media having various data structures stored thereon. The components can communicate via local or remote processes such as in accordance with a signal having one or more data packets (e.g., data from one component interacting with another component in a local system, distributed system, or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. As used herein, the term “and/or” is intended to have the same meaning as “or.” Moreover, articles “a” and “an” as used in the subject specification and annexed drawings should generally be construed to mean “one or more” unless specified otherwise or clear from context to be directed to a singular form. As used herein, the terms “example” or “exemplary” are utilized to mean serving as an example, instance, or illustration. For the avoidance of doubt, the subject matter disclosed herein is not limited by such examples. In addition, any aspect or design described herein as an “example” or “exemplary” is not necessarily to be construed as preferred or advantageous over other aspects or designs, nor is it meant to preclude equivalent exemplary structures and techniques known to those of ordinary skill in the art.

The herein disclosure describes non-limiting examples. For case of description or explanation, various portions of the herein disclosure utilize the term “each,” “every,” or “all” when discussing various examples. Such usages of the term “each,” “every,” or “all” are non-limiting. In other words, when the herein disclosure provides a description that is applied to “each,” “every,” or “all” of some particular object or component, it should be understood that this is a non-limiting example, and it should be further understood that, in various other examples, it can be the case that such description applies to fewer than “each,” “every,” or “all” of that particular object or component.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system, comprising:
a processor that executes computer-executable components stored in a non-transitory computer-readable memory, wherein the computer-executable components comprise:
a protocol component that infers, via execution of a first deep learning neural network, a prescribed imaging protocol that is to be performed by a medical imaging scanner on a medical patient;
a preparation component that infers, via execution of a second deep learning neural network on a preparation image or video of the medical patient that is captured by a camera associated with the medical imaging scanner, whether or not the medical patient is prepared for the prescribed imaging protocol; and
a guidance component that, in response to an inference that the medical patient is not prepared for the prescribed imaging protocol, initiates an electronic guidance action that explains or shows how to make the medical patient prepared for the prescribed imaging protocol.

2. The system of claim 1, wherein the guidance component, in response to an inference that the medical patient is prepared for the prescribed imaging protocol:
renders on a graphical user-interface of the medical imaging scanner a notification indicating that the prescribed imaging protocol is ready to be performed and requesting a user of the medical imaging scanner to approve performance of the prescribed imaging protocol; or
instructs the medical imaging scanner to perform the prescribed imaging protocol.

3. The system of claim 1, wherein the first deep learning neural network is a large language model that:
receives as input a textual prescription written by a medical professional attending to the medical patient; and
produces as output synthesized text indicating the prescribed imaging protocol in terms of anatomy or laterality.

4. The system of claim 1, wherein the first deep learning neural network is an image classifier that:
receives as input the preparation image or video of the medical patient captured by the camera; and
produces as output a classification label indicating the prescribed imaging protocol.

5. The system of claim 1, wherein the second deep learning neural network receives as input the preparation image or video and produces as output a localization indicating a current body pose or orientation of the medical patient, and wherein the preparation component infers that the medical patient is not prepared, in response to the current body pose or orientation not matching a requisite body pose or orientation specified in the prescribed imaging protocol.

6. The system of claim 1, wherein the second deep learning neural network receives as input the preparation image or video and produces as output a localization indicating a current scanner coil position on the medical patient, and wherein the preparation component infers that the medical patient is not prepared, in response to the current scanner coil position not matching a requisite scanner coil position specified in the prescribed imaging protocol.

7. The system of claim 1, wherein the electronic guidance action comprises rendering, on a graphical user-interface of the medical imaging scanner:
a current body pose or orientation or a current scanner coil position of the medical patient, inferred by the second deep learning neural network; and
a requisite body pose or orientation or a requisite scanner coil position, specified in the prescribed imaging protocol.

8. The system of claim 1, wherein a current scanner coil location of the medical patient inferred by the second deep learning neural network does not match a requisite scanner coil position specified in the prescribed imaging protocol, and wherein the electronic guidance action comprises shining a light or laser associated with the medical imaging scanner onto the body of the medical patient in accordance with the requisite scanner coil position.

9. The system of claim 1, wherein the camera is located in a separate room than the medical imaging scanner.

10. A computer-implemented method, comprising:
inferring, by a device operatively coupled to a processor and via execution of a first deep learning neural network, a prescribed imaging protocol that is to be performed by a medical imaging scanner on a medical patient;
inferring, by the device and via execution of a second deep learning neural network on a preparation image or video of the medical patient that is captured by a camera associated with the medical imaging scanner, whether or not the medical patient is prepared for the prescribed imaging protocol; and
initiating, by the device and in response to an inference that the medical patient is not prepared for the prescribed imaging protocol, an electronic guidance action that explains or shows how to make the medical patient prepared for the prescribed imaging protocol.

11. The computer-implemented method of claim 10, further comprising:
rendering, by the device, in response to an inference that the medical patient is prepared for the prescribed imaging protocol, and on a graphical user-interface of the medical imaging scanner, a notification indicating that the prescribed imaging protocol is ready to be performed and requesting a user of the medical imaging scanner to approve performance of the prescribed imaging protocol; or
instructing, by the device, the medical imaging scanner to perform the prescribed imaging protocol.

12. The computer-implemented method of claim 10, wherein the first deep learning neural network is a large language model that:
receives as input a textual prescription written by a medical professional attending to the medical patient; and
produces as output synthesized text indicating the prescribed imaging protocol in terms of anatomy or laterality.

13. The computer-implemented method of claim 10, wherein the first deep learning neural network is an image classifier that:
receives as input the preparation image or video of the medical patient captured by the camera; and
produces as output a classification label indicating the prescribed imaging protocol.

14. The computer-implemented method of claim 10, wherein the second deep learning neural network receives as input the preparation image or video and produces as output a localization indicating a current body pose or orientation of the medical patient, and wherein the device infers that the medical patient is not prepared, in response to the current body pose or orientation not matching a requisite body pose or orientation specified in the prescribed imaging protocol.

15. The computer-implemented method of claim 10, wherein the second deep learning neural network receives as input the preparation image or video and produces as output a localization indicating a current scanner coil position on the medical patient, and wherein the device infers that the medical patient is not prepared, in response to the current scanner coil position not matching a requisite scanner coil position specified in the prescribed imaging protocol.

16. The computer-implemented method of claim 10, wherein the electronic guidance action comprises rendering, on a graphical user-interface of the medical imaging scanner:
a current body pose or orientation or a current scanner coil position of the medical patient, inferred by the second deep learning neural network; and
a requisite body pose or orientation or a requisite scanner coil position, specified in the prescribed imaging protocol.

17. The computer-implemented method of claim 10, wherein a current scanner coil location of the medical patient inferred by the second deep learning neural network does not match a requisite scanner coil position specified in the prescribed imaging protocol, and wherein the electronic guidance action comprises shining a light or laser associated with the medical imaging scanner onto the body of the medical patient in accordance with the requisite scanner coil position.

18. The computer-implemented method of claim 10, wherein the camera is located in a separate room than the medical imaging scanner.

19. A computer program product for facilitating camera-based deep learning prediction and guidance for medical imaging protocols, the computer program product comprising a non-transitory computer-readable memory having program instructions embodied therewith, the program instructions executable by a processor to cause the processor to:
infer, via execution of a first deep learning neural network on a physician prescription corresponding to a medical patient or on a video feed depicting the medical patient, a prescribed imaging protocol that is to be performed by a magnetic resonance imaging (MRI) scanner on the medical patient;
infer, via execution of a second deep learning neural network on the video feed, whether or not an MRI coil position on the medical patient fails to match a requisite MRI coil position specified in the prescribed imaging protocol;
cause, in response to an inference that the MRI coil position does not match the requisite MRI coil position, an actuatable light or laser associated with the MRI scanner to shine onto the body of the medical patient, thereby visibly lighting the requisite MRI coil position on the medical patient.

20. The computer program product of claim 19, wherein the program instructions are further executable to cause the processor to:
cause, in response to an inference that the MRI coil position does match the requisite MRI coil position, the MRI scanner to perform the prescribed imaging protocol.
